SEQUENCE LISTING (42 sequences)

SEQ ID NO: 1 - DNA, 64 bp, Artificial Sequence
  Synthetic construct
ggagaaccaccttgttggcgtaagtctcatatttcaccgtttaagagctatgctggaaacagca

SEQ ID NO: 2 - DNA, 72 bp, Artificial Sequence
  Synthetic construct
gtatcccttggagaaccaccttgttggtcgcccttcagcacgcacagtttaagagctatgctggaaacagca

SEQ ID NO: 3 - DNA, 37 bp, Artificial Sequence
  Synthetic construct
ctagtactcgagaaaaaaagcaccgactcggtgccac

SEQ ID NO: 4 - DNA, 25 bp, Artificial Sequence
  Synthetic construct
caccgcatcaccttcaccctctcca

SEQ ID NO: 5 - DNA, 25 bp, Artificial Sequence
  Synthetic construct
aaactggagagggtgaaggtgatgc

SEQ ID NO: 6 - DNA, 25 bp, Artificial Sequence
  Synthetic construct
caccgcgtgctgaagtcaagtttga

SEQ ID NO: 7 - DNA, 25 bp, Artificial Sequence
  Synthetic construct
aaactcaaacttgacttcagcacgc

SEQ ID NO: 8 - DNA, 24 bp, Artificial Sequence
  Synthetic construct
caccgtcgacaggtaatggttgtc

SEQ ID NO: 9 - DNA, 24 bp, Artificial Sequence
  Synthetic construct
aaacgacaaccattacctgtcgac

SEQ ID NO: 10 - DNA, 24 bp, Artificial Sequence
  Synthetic construct
caccgcggtggacaatggaaggcc

SEQ ID NO: 11 - DNA, 24 bp, Artificial Sequence
  Synthetic construct
aaacggccttccattgtccaccgc

SEQ ID NO: 12 - DNA, 200 bp, Artificial Sequence
  Synthetic construct
gtgctggtttgcgtcgtagtctcctgcagcgtctggggtttccgttgcagtcctcggaaccaggacctcggcgtggcctagcgagttatggcgacgaaggtcgtgtgcgtgctgaagggcgacggcccagtgcagggcatcatcaatttcgagcagaaggcaagggctgggacggaggcttgtttgcgaggccgctccca

SEQ ID NO: 13 - DNA, 40 bp, Artificial Sequence
  Synthetic construct
aaagtgccacctgacaggtctggcctataaagtagtcgcg

SEQ ID NO: 14 - DNA, 35 bp, Artificial Sequence
  Synthetic construct
agctggagaccgtttgacccgctcctagcaaaggt

SEQ ID NO: 15 - DNA, 46 bp, Artificial Sequence
  Synthetic construct
aaagtgccacctgacattcttctaccagtcccaaacaaaagctctc

SEQ ID NO: 16 - DNA, 42 bp, Artificial Sequence
  Synthetic construct
agctggagaccgtttagcaaatgtcaatcccaaagttgggag

SEQ ID NO: 17 - DNA, 44 bp, Artificial Sequence
  Synthetic construct
aaagtgccacctgacctggttagccagttttcactgttctctgt

SEQ ID NO: 18 - DNA, 46 bp, Artificial Sequence
  Synthetic construct
agctggagaccgtttgcattttggaaagtcaagaggagagaattgc

SEQ ID NO: 19 - DNA, 23 bp, Artificial Sequence
  Synthetic construct
gcatgactttttcaagagtgcca

SEQ ID NO: 20 - DNA, 50 bp, Artificial Sequence
  Synthetic construct
acacgttcagagttctacagtccgacgatcgacgggacctacaagacgcg

SEQ ID NO: 21 - DNA, 50 bp, Artificial Sequence; identical to SEQ ID NO: 20
  Synthetic construct

SEQ ID NO: 22 - DNA, 50 bp, Artificial Sequence
  Synthetic construct
aatgatacggcgaccaccgagatctacacgttcagagttctacagtccga

SEQ ID NO: 23 - DNA, 64 bp, Artificial Sequence
  Synthetic construct
caagcagaagacggcatacgagataaacagtgtgactggagttccttggcacccgagaattcca

SEQ ID NO: 24 - DNA, 64 bp, Artificial Sequence
  Synthetic construct
caagcagaagacggcatacgagataaaccccgtgactggagttccttggcacccgagaattcca

SEQ ID NO: 25 - DNA, 64 bp, Artificial Sequence
  Synthetic construct
caagcagaagacggcatacgagataaacggcgtgactggagttccttggcacccgagaattcca

SEQ ID NO: 26 - DNA, 34 bp, Artificial Sequence
  Synthetic construct
ctccaccaggtgaaatatgagacttacgcaacat

SEQ ID NO: 27 - DNA, 34 bp, Artificial Sequence
  Synthetic construct
atgttgagtaagtctcatatttcacctggtggag

SEQ ID NO: 28 - DNA, 34 bp, Artificial Sequence
  Synthetic construct
gaagccgggccttccattgtccaccgcaaatgct

SEQ ID NO: 29 - DNA, 34 bp, Artificial Sequence
  Synthetic construct
agcatttgcggtggacaatggaaggcccggcttc

SEQ ID NO: 30 - DNA, 34 bp, Artificial Sequence
  Synthetic construct
gaaggccgtggcgtgctgctgaagggcgacggcc

SEQ ID NO: 31 - DNA, 32 bp, Artificial Sequence
  Synthetic construct
ggccgtcgcccttcagcacgcacacggccttc

SEQ ID NO: 32 - DNA, 34 bp, Artificial Sequence
  Synthetic construct
gaaggtcgtgtgtgcgtgctgaagggcgacggcc

SEQ ID NO: 33 - DNA, 87 bp, Artificial Sequence
  Synthetic construct
gttggaaccattcaaaacagcatagcaagttaaaataaggctagtccgttatcaacttgaaaaagtggcaccgagtcggtgcttttt

SEQ ID NO: 34 - DNA, 74 bp, Artificial Sequence
  Synthetic construct
aagaaatttaaaaagggactaaaataaagagtttgcgggactctgcggggttacaatcccctaaaaccgctttt

SEQ ID NO: 35 - DNA, 96 bp, Artificial Sequence
  Synthetic construct
atctaaaattataaatgtaccaaataattaatgctctgtaatcatttaaaagtattttgaacggacctctgtttgacacgtctgaataactaaaaa

SEQ ID NO: 36 - DNA, 109 bp, Artificial Sequence
  Synthetic construct
tgtaagggacgccttacacagttacttaaatcttgcagaagctacaaagataaggcttcatgccgaaatcaacaccctgtcattttatggcagggtgttttcgttattt

SEQ ID NO: 37 - DNA, 96 bp, Artificial Sequence
  Synthetic construct
ttgtggtttgaaaccattcgaaacaacacagcgagttaaaataaggcttagtccgtactcaacttgaaaaggtggcaccgattcggtgtttttttt

SEQ ID NO: 38 - DNA, 76 bp, Artificial Sequence
  Synthetic construct
ccacaagccttggaattattcctgaactactctgtgactccaccaggtgaaatagctactaacttcagcctgctga

SEQ ID NO: 39 - DNA, 76 bp, Artificial Sequence
  Synthetic construct
gagtttgtaactgctgctgggattacacatggcatggatgagctctacaaatgagacttacgcaacatctgggctt

SEQ ID NO: 40 - DNA, 94 bp, Artificial Sequence
  Synthetic construct
gggctccattgtccaccgcaaatgcttcggtaccagcggcagcggagagggcagaggaagtcttctaacatgcggtgacgtggaggagaatccc

SEQ ID NO: 41 - DNA, 96 bp, Artificial Sequence
  Synthetic construct
gccgccgggatcactctcggcatggacgagctgtacaagtaatagggtaccactttcctgctcctctctgtctctagcacacaactgtgaatgtcc

SEQ ID NO: 42 - DNA, 96 bp, Artificial Sequence
  Synthetic construct
cctcggaaccaggacctcggcgtggcctagcgagttatggcgacgaaggtcgtgtgcgtgctgaagggcgacggcccagtgcagggcatcatcaat

(19) United States
(12) Patent Application Publication
(10) Pub. No.: US 2018/0016601 A1
(43) Pub. Date: Jan. 18, 2018

(54) Methods for Modulating Genome Editing

(71) Applicants: The Board of Trustees of the Leland Stanford Junior University, Palo Alto, CA (US); The J. David Gladstone Institutes, a Testamentary Trust established under the Will of J. David Glads, San Francisco, CA (US); The Regents of the University of California, Oakland, CA (US)

(72) Inventors: Lei S. Qi, Palo Alto, CA (US); Sheng Ding, Orinda, CA (US); Chen Yu, San Francisco, CA (US)

(21) Appl. No.: 15/649,304

(22) Filed: Jul. 13, 2017

Related U.S. Application Data
(63) Continuation of application No. PCT/US2016/013375, filed on Jan. 14, 2016.
(60) Provisional application No. 62/104,035, filed on Jan. 15, 2015.

(51) Int. Cl. C12N 15/90; C12Q 1/44; C12N 15/10; A61K 38/46; A61K 31/365; A61K 31/63; A61K 31/513; A61K 31/505; C12Q 1/68; C12N 9/22

(52) U.S. Cl. CPC C12N 15/907; C12Q 1/68; C12Q 1/44; C12N 15/1024; C12N 9/22; A61K 31/365; A61K 31/63; A61K 31/513; A61K 31/505; A61K 38/465

(57) ABSTRACT

Provided herein are methods and kits for modulating genome editing of target DNA. The invention includes using small molecules that enhance or repress homology-directed repair (HDR) and/or nonhomologous end joining (NHEJ) repair of double-strand breaks in a target DNA sequence. Also provided herein are methods for preventing or treating a genetic disease in a subject by enhancing precise genome editing to correct a mutation in a target gene associated with the genetic disease. Further provided herein are systems and methods for screening small molecule libraries to identify novel modulators of genome editing. The present invention can be used with any cell type and at any gene locus that is amenable to nuclease-mediated genome editing technology.

### CROSS-REFERENCES TO RELATED APPLICATIONS

The present application is a Continuation of PCT/US2016/013375 filed Jan. 14, 2016; which claims priority to U.S. Provisional Patent Application No. 62/104,035 filed Jan. 15, 2015; the disclosures which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support under Grant Nos. DP5OD017887, OD017887, and DA036858, awarded by the National Institutes of Health, and Grant No. U01HL107436, awarded by the National Heart, Lung and Blood Institute. The government has certain rights in the invention.

### BACKGROUND OF THE INVENTION

It has been discovered that bacteria and archaea utilize short RNA to target and direct degradation of foreign nucleic acids. This RNA-guided defense system, termed a clustered regularly interspaced short palindromic repeats (CRISPR/CRISPR-associated (Cas)) system involves acquiring and integrating targeting spacer sequences from the foreign DNA into the CRISPR locus, expressing and processing short guiding CRISPR RNAs containing spacer-repeat units, and cleaving DNA complementary to the spacer sequence to silence the foreign DNA. Recently, the CRISPR/Cas system has been adapted into a tool for targeted genome editing of cells and animal models. The nucleic acid-guided Cas nuclease can be used to induce double-strand breaks (DSBs) at a target genomic locus by specifying a short nucleotide sequence within its guide nucleic acid (e.g., DNA-targeting RNA).. Upon cleavage at the target locus, DNA damage repair can occur via the nonhomologous end joining (NHEJ) and/or homology-directed repair (HDR) pathway. In the absence of a repair template, the DSBs can re-ligate through NHEJ which leaves insertion/deletion (indel) mutations. Alternatively, in the presence of an exogenously introduced repair template, HDR can occur. The repair template can be a double-stranded DNA targeting construct with homology arms that flank the insertion site, or single-stranded oligonucleotides also with homology arms.

Although the CRISPR/Cas system is a highly specific and efficient method of genome engineering, it is prone to generating off-target modifications. Strategies for minimizing the occurrence of off-target DNA modification can include optimizing the concentration of Cas9 enzyme in the system, selecting target sequences with a minimum number of similar sequences in the target genome, and using a double nicking strategy to introduce double-strand breaks at the target site. There is a need in the art for a simple and efficient method for modulating HDR and/or NHEJ mediated repair in the CRISPR/Cas system as well as other nuclease-mediated methods. The present invention satisfies this and other needs.

### BRIEF SUMMARY OF THE INVENTION

The present invention provides methods and kits for modulating genome editing of target DNA. The invention includes using small molecules that enhance or repress homology-directed repair (HDR) and/or nonhomologous end joining (NHEJ) repair of double-strand breaks in a target DNA sequence. The present invention also provides methods for preventing or treating a disease in a subject by enhancing precise genome editing to correct a mutation in a target gene associated with the disease. The present invention further provides systems and methods for screening small molecule libraries to identify novel modulators of genome editing. The present invention can be used with any cell type and at any gene locus that is amenable to nuclease-mediated genome editing technology.

The methods, kits, and systems disclosed herein can be used in ex vivo therapy. Ex vivo therapy can comprise administering a composition (e.g., a cell) generated or modified outside of an organism to a subject (e.g., patient). In some embodiments, the composition (e.g., a cell) can be generated or modified by the methods disclosed herein. For example, the method to screen for a modulator of genome editing can be used to find a novel composition (e.g., small molecule) that can be used to enhance homologous recombination (e.g., in a CRISPR/Cas system), which in turn can be used in ex vivo therapy (e.g., modifying cells with the novel composition found through the screening methods). For example, ex vivo therapy can comprise administering a composition (e.g., a cell) generated or modified outside of an organism to a subject (e.g., patient).

In some embodiments, the composition (e.g., a cell) can be from the subject (e.g., patient) to be treated by ex vivo therapy. In some embodiments, ex vivo therapy can include cell-based therapy, such as adoptive immunotherapy.

In a first aspect, the present invention provides a method for modulating genome editing of a target DNA in a cell, the method comprising:
(a) introducing into the cell a DNA nuclease or a nucleotide sequence encoding the DNA nuclease, wherein the DNA nuclease is capable of creating a double-strand break in the target DNA to induce genome editing of the target DNA; and
(b) contacting the cell with a small molecule compound under conditions that modulate genome editing of the target DNA induced by the DNA nuclease.

In a second aspect, the present invention provides a kit comprising: (a) a DNA nuclease or a nucleotide sequence encoding the DNA nuclease; and (b) a small molecule compound that modulates genome editing of a target DNA in a cell.

In a third aspect, the present invention provides a method for preventing or treating a genetic disease in a subject, the method comprising:
(a) administering to the subject a DNA nuclease or a nucleotide sequence encoding the DNA nuclease in a sufficient amount to correct a mutation in a target gene associated with the genetic disease; and
(b) administering to the subject a small molecule compound in a sufficient amount to enhance the effect of the DNA nuclease.

In a fourth aspect, the present invention provides a system for identifying a small molecule compound for modulating genome editing of a target DNA in a cell, the system comprising:
(a) a first recombinant expression vector comprising a nucleotide sequence encoding a Cas9 polypeptide or a variant thereof;
(b) a second recombinant expression vector comprising a nucleotide sequence encoding a DNA-targeting RNA operably linked to a promoter, wherein the nucleotide sequence comprises:
   (i) a first nucleotide sequence that is complementary to the target DNA; and
   (ii) a second nucleotide sequence that interacts with the Cas9 polypeptide or the variant thereof; and
(c) a recombinant donor repair template comprising:
   (i) a reporter cassette comprising a nucleotide sequence encoding a reporter polypeptide operably linked to a nucleotide sequence encoding a self-cleaving peptide; and
   (ii) two nucleotide sequences comprising two non-overlapping, homologous portions of the target DNA, wherein the nucleotide sequences are located at the 5′ and 3′ ends of the reporter cassette.

In a fifth aspect, the present invention provides a kit comprising the system described above and an instruction manual.

In a sixth aspect, the present invention provides a method for identifying a small molecule compound for modulating genome editing of a target DNA in a cell, the method comprising:
(a) introducing into a cell:
   (i) a first recombinant expression vector comprising a nucleotide sequence encoding a Cas9 polypeptide or a variant thereof,
   (ii) a second recombinant expression vector comprising a nucleotide sequence encoding a DNA-targeting RNA operably linked to a promoter, wherein the nucleotide sequence comprises a first nucleotide sequence that is complementary to a target DNA and a second nucleotide sequence that interacts with the Cas9 polypeptide or the variant thereof, and
   (iii) a recombinant donor repair template comprising a reporter cassette comprising a nucleotide sequence encoding a reporter polypeptide operably linked to a nucleotide sequence encoding a self-cleaving peptide, and two nucleotide sequences comprising two non-overlapping, homologous portions of the target DNA, wherein the nucleotide sequences are located at the 5′ and 3′ ends of the reporter cassette,
to generate a modified cell;
(b) contacting the modified cell with a small molecule compound;
(c) detecting the level of the reporter polypeptide in the modified cell; and
(d) determining that the small molecule compound modulates genome editing if the level of the reporter polypeptide is increased or decreased compared to its level prior to step (b).

In another aspect, provided herein is a method to screen for a modulator of genome editing comprising: (a) contacting a cell undergoing nuclease-mediated genome editing with a small molecule compound; and (b) comparing efficiency of the nuclease-mediated genome editing of a target DNA sequence in the contacted cell to a control cell that has not been contacted with the small molecule compound, wherein the small molecule compound enhances the efficiency of the nuclease-mediated genome editing by at least 1.1 fold. In some embodiments, the modulator of genome editing can be used to increase efficiency of genome editing. In some cases, the modulator of genome editing can be used to decrease cellular toxicity.

In some embodiments, the method to screen for a modulator of genome editing can be used in ex vivo therapy. For example, the method to screen for a modulator of genome editing can be used to find a novel composition (e.g., small molecule) that can be used to enhance homologous recombination (e.g., in a CRISPR/Cas system), which in turn can be used in ex vivo therapy (e.g., modifying cells with the novel composition found through the screening methods). Ex vivo therapy can comprise administering a composition (e.g., a cell) generated or modified outside of an organism to a subject (e.g., patient). In some embodiments, the composition (e.g., a cell) is generated or modified by the method disclosed herein. In some embodiments, the composition (e.g., a cell) can be derived from the subject (e.g., patient) to be treated by the ex vivo therapy. In some embodiments, ex vivo therapy can include cell-based therapy, such as adoptive immunotherapy.

In some embodiments, the composition used in ex vivo therapy can be a cell. The cell can be a primary cell, including but not limited to, peripheral blood mononuclear cells (PBMC), peripheral blood lymphocytes (PBL), and other blood cell subsets. The cell can be an immune cell. The cell can be a T cell, a natural killer cell, a monocyte, a natural killer T cell, a monocyte-precursor cell, a hematopoietic stem cell or a non-pluripotent stem cell, a stem cell, or a progenitor cell. The cell can be a hematopoietic progenitor cell. The cell can be a human cell. The cell can be selected. The cell can be expanded ex vivo. The cell can be expanded in vivo. The cell can be CD45RO(−), CCR7(+), CD45RA(+), CD62L(+), CD27(+), CD28(+), or IL-7Ra(+). The cell can be autologous to a subject in need thereof. The cell can be non-autologous to a subject in need thereof. The cell can be a good manufacturing practices (GMP) compatible reagent. The cell can be a part of a combination therapy to treat diseases, including cancer, infections, autoimmune disorders, or graft-versus-host disease (GVHD), in a subject in need thereof.

In some embodiments, the small molecule compound can enhance homology-directed repair (HDR) efficiency and/or can enhance nonhomologous end joining (NHEJ) efficiency of the nuclease-mediated genome editing. In some cases, the nuclease-mediated genome editing can use a nuclease selected from a CRISPR-associated protein (Cas) polypeptide, a zinc finger nuclease (ZFN), a transcription activator-like effector nuclease (TALEN), a meganuclease, a variant thereof, a fragment thereof, or any combination thereof. If the Cas polypeptide is used, the Cas polypeptide can be a Cas9 polypeptide, a variant thereof, or a fragment thereof. In some embodiments, the nuclease-mediated genome editing can use a CRISPR/Cas system.

In some embodiments, the method of (a) can further comprise contacting the cell with a recombinant donor repair template. In some cases, the method of (a) can further comprise contacting the cell with a nucleic acid, e.g., a DNA-targeting RNA, or a nucleotide sequence encoding the guide nucleic acid (e.g., DNA-targeting RNA). In some cases, the method of (a) can further comprise contacting the cell with a DNA replication enzyme inhibitor. In some cases, the DNA replication enzyme inhibitor is selected from a DNA ligase inhibitor, a DNA gyrase inhibitor, a DNA helicase inhibitor, or any combination thereof.

In some embodiments, contacting the cell with a combination of the small molecule compound and the DNA replication enzyme inhibitor can enhance efficiency of the nuclease-mediated genome editing compared to contacting the cell with either the small molecule compound or the DNA replication enzyme inhibitor. In some cases, the at least one component of the nuclease-mediated genome editing can be introduced into the cell using a delivery system selected from a nanoparticle, a liposome, a micelle, a virosome, a nucleic acid complex, a transfection agent, an electroporation agent, a nucleofection agent, a lipofection agent or any combination thereof. In some embodiments, the small molecule compound is selected from a β adrenoceptor agonist, Brefeldin A, nucleoside, a derivative thereof, an analog thereof, or any combination thereof. In some cases, the small molecule compound can be at a concentration of about 0.01 μM to about 10 μM, e.g., about 0.01 μM to about 0.05 μM, about 0.01 μM to about 0.1 μM, about 0.01 μM to about 0.2 μM, about 0.01 μM to about 0.4 μM, about 0.01 μM to about 0.6 μM, about 0.01 μM to about 0.8 μM, about 0.01 μM to about 1 μM, about 0.01 μM to about 2 μM, about 0.01 μM to about 3 μM, about 0.01 μM to about 4 μM, about 0.01 μM to about 5 μM, about 0.01 μM to about 6 μM, about 0.01 μM to about 7 μM, about 0.01 μM to about 8 μM, about 0.01 μM to about 9 μM, about 0.1 μM to about 1 μM, about 0.1 μM to about 2 μM, about 0.1 μM to about 3 μM, about 0.1 μM to about 4 μM, about 0.1 μM to about 5 μM, about 0.1 μM to about 6 μM, about 0.1 μM to about 7 μM, about 0.1 μM to about 8 μM, about 0.1 μM to about 9 μM, about 0.1 μM to about 10 μM, about 0.5 μM to about 1 μM, about 0.5 μM to about 2 μM, about 0.5 μM to about 4 μM, about 0.5 μM to about 6 μM, about 0.5 μM to about 8 μM, about 0.5 μM to about 10 μM, about 1μM to about 2 μM, about 1 μM to about 4 μM, about 1 μM to about 6 μM, about 1 μM to about 8 μM, about 1 μM to about 10 μM, about 2 μM to about 4 μM, about 2 μM to about 6 μM, about 2 μM to about 8 μM, about 2 μM to about 10 μM, about 4 μM to about 6 μM, about 4 μM to about 8 μM, about 4μM to about 10 μM, about 6 μM to about 8 μM, about 6 μM to about 10 μM, or about 8 μM to about 10 μM. In some cases, the cell is contacted with the small molecule compound for about 2, 4, 6, 8, 10, 12, 24, 36, 48, 60, or 72 hours.

In some embodiments, the cell is selected from a stem cell, human cell, mammalian cell, non-mammalian cell, vertebrate cell, invertebrate cell, plant cell, eukaryotic cell, bacterial cell, immune cell, T cell, or archaeal cell. In some cases, the method can further comprise isolating, selecting, culturing, and/or expanding the cell.

In another aspect, provided herein is a modulator of nuclease-mediated genome editing of a target DNA sequence, comprising a small molecule compound identified using any one of the methods as described.

Other objects, features, and advantages of the present invention will be apparent to one of skill in the art from the following detailed description and figures.

### BRIEF DESCRIPTION OF THE DRAWINGS

FIGS. 1A-1G show the establishment of a high-throughput chemical screening platform for modulating CRISPR-mediated HDR efficiency. FIG. 1A illustrates a fluorescence reporter system in E14 mouse ES cells to characterize the HDR efficiency. An sfGFP-encoding template was inserted at the Nanog locus (5′-CTCCACCAGGTGAAATATGAGACTTACGCAACAT-3′ (SEQ ID NO:26); 5′-ATGTTGAGTAAGTCTCATATTTCACCTGGTGGAG-3′ (SEQ ID NO:27)). The sgRNA target site including the stop codon (TGA) is shaded in grey. The cutting site (scissors) is 3 bp downstream of CCA in this case. Binding sites of two sets of primers are shown by arrows. Primer set #1 binds to the sequences outside of the homology arms, and primer set #2 contains a forward primer binding to the sfGFP sequence and a reverse primer binding outside of the 3′ homology arm. FIG. 1B shows fluorescence histograms of mouse ES cells transfected with different plasmid combinations using flow cytometry analysis. FIG. 1C shows sequencing results of the Nanog locus in GFP-positive cells. FIG. 1D presents a scheme of the chemical screening platform and a waterfall plot of 3,918 small molecules screened for their activity of CRISPR-mediated gene insertion. Highlighted dots are validated compounds that showed increased or decreased insertion efficiency. The dotted line showed the mean value of all screened compounds. FIG. 1E illustrates the validation of two enhancing and two repressing compounds using flow cytometry analysis. FIG. 1F shows the efficiency of sfGFP insertion into the Nanog locus. Gel pictures show sfGFP tagging using two sets of primers as shown in FIG. 1A. FIG. 1G shows dose-dependent effects of four compounds for modulating CRISPR gene editing. All data were normalized to the knock-in efficiency of DMSO treated cells (dotted lines). Error bars represent the standard deviation of three biological replicates.

FIGS. 2A-2G show that different identified small molecules can enhance HDR or NHEJ-mediated CRISPR genome editing. FIG. 2A shows a scheme of insertion strategy at the human ACTA2 locus (5′-GAAGCCGGGCCTTCCATTGTCCACCGCAAATGCT-3′ (SEQ ID NO: 28); 5′-AGCATTTGCGGTGGACAATGGAAGGCCCGGCTTC-3′ (SEQ ID NO: 29)). The single guide RNA (sgRNA) target site is shaded in grey. FIG. 2B shows sequencing results of the ACTA2 locus in Venus-positive HeLa cells. FIG. 2C illustrates the efficiency of Venus insertion measured by flow cytometry analysis. The error bars indicate the standard deviation of three samples, and the p values are calculated using two-tailed student t-test (*, p<0.05; **, p<0.01). FIG. 2D provides the strategy for introducing the A4V point mutation at the human SOD1 locus in human iPS cells (5′-GAAGGCCGTGGCGTGCTGCTGAAGGGCGACGGCC-3′ (SEQ ID NO:30); 5′-GGCCGTCGCCCTTCAGCACGCACACGGCCTTC-3′ (SEQ ID NO: 31); 5′-GAAGGTCGTGTGTGCGTGCTGAAGGGCGACGGCC-3′ (SEQ ID NO: 32)). The sgRNA target site is shaded in grey. FIG. 2E shows sequencing results of the SOD1 locus. FIG. 2F provides a comparison of A4V allele mutant frequency and indel allele frequency in human iPS cells assayed by PCR cloning and bacterial colony sequencing with no template, DMSO or L755507. FIG. 2G shows testing of knockout efficiency using a clonal mouse ES cell line carrying a monoallelic sfGFP insertion at the Nanog locus in the presence of L755705 and AZT. The dot plots of cells transfected with a non-cognate sgRNA (sgGAL4) is shown on the top. The panel shows cells transfected with three different sgRNAs (their target sites shown in the scheme) in the presence of DMSO (left), L755507 (middle), and AZT (right).

FIGS. 3A-3E show the high-throughput chemical screening platform for modulating CRISPR-mediated HDR efficiency. FIG. 3A provides a fluorescence histogram of mouse ES cells transfected with Cas9, sgNanog, and/or a control template containing p2A-sfGFP without the homology arms (HAs). FIG. 3B shows a scheme of the high-throughput chemical screening platform. FIG. 3C provides a characterization of GFP insertion efficiency at the Nanog locus in mouse ES cells with different treatment windows of four small molecules. FIG. 3D illustrates cell number at day 3 after post electroporation. Cells were treated with small molecules at the first 24 hours. FIG. 3E shows cell viability as measured by the MTS assay (Promega). Absorbance at 490 nm was normalized to E14 cells. In FIGS. 3C-3E, error bars represent the standard deviation of three biological replicates.

FIGS. 4A-4G illustrate the use of Nanog-sfGFP mouse ES cells to identify small molecules that modulated CRISPR-mediated genetic editing. FIG. 4A provides a scheme of generating a clonal mouse ES cell line carrying a monoallelic sfGFP insertion at the Nanog locus. Two sets of primer binding sites are shown by arrows. One primer set (#1) binds to the sequences outside of the homologous arms, and the other primer set (#2) contains a forward primer binding to the sfGFP sequence and a reverse primer binding outside of the 3′ homologous arm. FIG. 4B provides a gel picture showing validation of single allele tagging using two sets of primers. FIG. 4C shows immunofluorescence of Oct4 and Sox2 of E14 cells treated with small molecules after 10 passages. Cells were treated with small molecules for the first 24 hours after splitting. FIG. 4D shows flow cytometry analysis of Nanog of E14 cells treated with small molecules. FIG. 4E provides microscopic images of Nanog-sfGFP ES cells electroporated with different sgRNAs. FIG. 4F provides microscopic images of Nanog-sfGFP mouse ES cells electroporated with sgsfGFP-1 in the presence of DMSO, L755507 (5 μM), or AZT (1 μM). FIG. 4G shows microscopic images of Nanog-sfGFP mouse ES cells treated with AZT for 10 passages. Cells were treated with small molecules for the first 24 hours after each splitting. Scale bars represent 50 μm.

FIG. 5 provides deep sequencing analysis of sfGFP targeting sgGFP-2.

FIG. 6 shows the efficiency of homologous-directed repair (HDR) using a combination of a DNA ligase inhibitor (“SCR7a”) and a β3-adrenergic receptor agonist (“L755507”) compared to the efficiency of HDR using either compound alone.

### DETAILED DESCRIPTION OF THE INVENTION

### I. INTRODUCTION

Provided herein are methods and kits for modulating genome editing of target DNA. The invention includes using small molecules that enhance or repress homology-directed repair (HDR) or nonhomologous end joining (NHEJ) repair of double-strand breaks in a target DNA sequence. Also provided herein are methods for preventing or treating a disease, e.g., a genetic disease, in a subject by enhancing precise genome editing to correct a mutation in a target gene associated with the genetic disease. Also provided herein are methods for preventing or treating a disease (e.g. cancer) in a subject by enhancing precise genome editing for genetically modifying cells and nucleic acids for therapeutic applications. Further provided herein are systems and methods for screening small molecule libraries to identify novel modulators of genome editing. The present invention can be used with any cell type and at any gene locus that is amenable to nuclease-mediated genome editing technology.

### II. GENERAL

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### III. DEFINITIONS

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “genome editing” refers to a type of genetic engineering in which DNA is inserted, replaced, or removed from a target DNA, e.g., the genome of a cell, using one or more nucleases and/or nickases. The nucleases create specific double-strand breaks (DSBs) at desired locations in the genome, and harness the cell's endogenous mechanisms to repair the induced break by homology-directed repair (HDR) (e.g., homologous recombination) or by nonhomologous end joining (NHEJ). The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In particular embodiments, nuclease-mediated genome editing of a target DNA sequence can be “modulated” (e.g., enhanced or inhibited) using the small molecule compounds described herein alone or in combination with DNA replication enzyme inhibitors, e.g., to improve the efficiency of precise genome editing via homology-directed repair (HDR).

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “nonhomologous end joining” or “NHEJ” refers to a pathway that repairs double-strand DNA breaks in which the break ends are directly ligated without the need for a homologous template.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” or “nucleotide sequence encoding a polypeptide” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

A “recombinant expression vector” is a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector.

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

A “reporter cassette” refers to a polynucleotide comprising a promoter or other regulatory sequence operably linked to a sequence encoding a reporter polypeptide.

The term “single nucleotide polymorphism” or “SNP” refers to a change of a single nucleotide with a polynucleotide, including within an allele. This can include the replacement of one nucleotide by another, as well as deletion or insertion of a single nucleotide. Most typically, SNPs are biallelic markers although tri- and tetra-allelic markers can also exist. By way of non-limiting example, a nucleic acid molecule comprising SNP A\C may include a C or A at the polymorphic position.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent (e.g., DNA nuclease, small molecule compound, etc.) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

The term “pharmaceutically acceptable carrier” refers to a substance that aids the administration of an agent (e.g., DNA nuclease, small molecule compound, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical carriers are useful in the present invention.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

### IV. DESCRIPTION OF THE EMBODIMENTS

In a first aspect, the present invention provides a method for modulating genome editing of a target DNA in a cell, the method comprising:
(a) introducing into the cell a DNA nuclease or a nucleotide sequence encoding the DNA nuclease, wherein the DNA nuclease is capable of creating a double-strand break in the target DNA to induce genome editing of the target DNA; and
(b) contacting the cell with a small molecule compound under conditions that modulate genome editing of the target DNA induced by the DNA nuclease.

In some embodiments, the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof. In certain instances, the Cas polypeptide is a Cas9 polypeptide, a variant thereof, or a fragment thereof.

In some embodiments, step (a) of the method further comprises introducing into the cell a guide nucleic acid, e.g., DNA-targeting RNA (e.g., a single guide RNA or sgRNA or a double guide nucleic acid) or a nucleotide sequence encoding the guide nucleic acid (e.g., DNA-targeting RNA). In certain instances, the DNA-targeting RNA comprises at least two different DNA-targeting RNAs, wherein each DNA-targeting RNA is directed to a different target DNA.

In some embodiments, the small molecule compound that modulates genome editing is selected from the group consisting of a β adrenoceptor agonist or an analog thereof, Brefeldin A or an analog thereof, a nucleoside analog, a derivative thereof, and a combination thereof.

In some embodiments, the small molecule compound enhances or inhibits genome editing of the target DNA compared to a control cell that has not been contacted with the small molecule compound.

In some embodiments, the genome editing comprises homology-directed repair (HDR) of the target DNA. In certain embodiments, step (a) of the method further comprises introducing into the cell a recombinant donor repair template. In some instances, the recombinant donor repair template comprises two nucleotide sequences comprising two non-overlapping, homologous portions of the target DNA, wherein the nucleotide sequences are located at the 5′ and 3′ ends of a nucleotide sequence corresponding to the target DNA to undergo genome editing. In other instances, the recombinant donor repair template comprises a synthetic single-stranded oligodeoxynucleotide (ssODN) template, and two nucleotide sequences comprising two non-overlapping, homologous portions of the target DNA, wherein the nucleotide sequences are located at the 5′ and 3′ ends of nucleotide sequence encoding the mutation. In particular embodiments, the small molecule compound that enhances HDR is a _{R} adrenoceptor agonist (e.g., L755507), Brefeldin A, a derivative thereof, an analog thereof, or a combination thereof.

In particular embodiments, the small molecule compound that inhibits HDR is a nucleoside analog (e.g., azidothymidine (AZT), trifluridine (TFT), etc.), a derivative thereof, or a combination thereof.

In other embodiments, the genome editing comprises nonhomologous end joining (NHEJ) of the target DNA. In particular embodiments, the small molecule compound that enhances NHEJ is a nucleoside analog (e.g., azidothymidine (AZT)) or a derivative thereof. In particular embodiments, the small molecule compound that inhibits NHEJ is a _{R} adrenoceptor agonist (e.g., L755507), a derivative thereof, or an analog thereof.

In certain embodiments, the small molecule compound enhances the efficiency of HDR of the target DNA and decreases the efficiency of NHEJ of the target DNA. A non-limiting example of such a small molecule compound is L755507. In certain other embodiments, the small molecule compound enhances the efficiency of NHEJ of the target DNA and decreases the efficiency of HDR of the target DNA. A non-limiting example of such a small molecule compound is azidothymidine (AZT).

In some embodiments, step (b) of the method further comprises contacting the cell with a DNA replication enzyme inhibitor. In certain instances, the DNA replication enzyme inhibitor is selected from the group consisting of a DNA ligase inhibitor, a DNA gyrase inhibitor, a DNA helicase inhibitor, and a combination thereof. Non-limiting examples of DNA ligase inhibitors include compounds that inhibit one or more types of DNA ligases (I, III, IV) such as Scr7 (5,6-bis((E)-benzylideneamino)-2-thioxo-2,3 -dihydropyrimidin-4(1H)-one; CAS 159182-43-1), L189 (6-amino-2,3-dihydro-5-[(phenylmethylene)amino]-2-4(1H)-pyrimidineone; CAS 64232-83-3), derivatives thereof, analogs thereof, and combinations thereof. Non-limiting examples of DNA gyrase inhibitors include quinolones (e.g., nalidixic acid), fluoroquinolones (e.g., ciprofloxacin), coumarins (e.g., novobiocin), cyclothialidines, CcdB toxin, microcin B17, derivatives thereof, analogs thereof, and combinations thereof. Non-limiting examples of DNA helicase inhibitors include ML216 (N-[4-fluoro-3 -(trifluoromethyl)phenyl]-N′-[5 -(4-pyridinyl)-1,3,4-thiadiazol-2-yl]-urea; CAS 1430213-30-1), NSC 19630 (1-(propoxymethyl)-maleimide; CAS 72835-26-8), dibenzothiepins, derivatives thereof, analogs thereof, and combinations thereof.

In some embodiments, a combination of the small molecule compound and the DNA replication enzyme inhibitor enhances or inhibits genome editing of the target DNA compared to a control cell that has been contacted with either the small molecule compound or the DNA replication enzyme inhibitor. In certain embodiments, a combination of the small molecule compound and the DNA replication enzyme inhibitor enhances homology-directed repair (HDR) of the target DNA. In particular embodiments, the combination comprises a β adrenoceptor agonist (e.g., L755507) or a derivative or analog thereof and a DNA ligase inhibitor (e.g., Scr7) or a derivative or analog thereof.

In some embodiments, the cell is contacted with the small molecule compound at a concentration of about 0.1 μM to about 10 μM. In other embodiments, the cell is contacted with the small molecule compound for about 24 hours. In other embodiments, the cell is contacted with the small molecule compound for about 2, 4, 6, 8, 10, 12, 24, 36, 48, 60, or 72 hours. For example, the cell can be contacted with the small molecule compound for about 2 to about 4; about 4 to about 6; about 6 to about 8; about 8 to about 10; about 10 to about 12; about 12 to about 18; about 18 to about 24; about 2 to about 24; about 24 to about 36; about 36 to about 48; about 48 to about 60; or about 60 to about 72 hours. In certain embodiments, the cell is selected from the group consisting of a stem cell, human cell, mammalian cell, non-mammalian cell, vertebrate cell, invertebrate cell, plant cell, eukaryotic cell, bacterial cell, immune cell, T cell, and archaeal cell. In certain other embodiments, the method further comprises: (c) isolating, selecting, culturing, and/or expanding the cell.

In a second aspect, the present invention provides a kit comprising: (a) a DNA nuclease or a nucleotide sequence encoding the DNA nuclease; and (b) a small molecule compound that modulates genome editing of a target DNA in a cell.

In some embodiments, the kit further comprises one or more of the following components: a guide nucleic acid (e.g., DNA-targeting RNA) or a nucleotide sequence encoding the guide nucleic acid (e.g., DNA-targeting RNA); a recombinant donor repair template; and a DNA replication enzyme inhibitor.

In a third aspect, the present invention provides a method for preventing or treating a genetic disease in a subject, the method comprising:
(a) administering to the subject a DNA nuclease or a nucleotide sequence encoding the DNA nuclease in a sufficient amount to correct a mutation in a target gene associated with the genetic disease; and
(b) administering to the subject a small molecule compound in a sufficient amount to enhance the effect of the DNA nuclease.

In some embodiments, the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation disease or disorders, inflammation, immune-related diseases or disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases and disorders.

In some embodiments, the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof. In certain instances, the Cas polypeptide is a Cas9 polypeptide, a variant thereof, or a fragment thereof

In some embodiments, step (a) of the method further comprises administering to the subject a recombinant donor repair template. In other embodiments, step (a) of the method further comprises administering to the subject a DNA-targeting RNA or a nucleotide sequence encoding the DNA-targeting RNA.

In some embodiments, the small molecule compound is selected from the group consisting of a β adrenoceptor agonist (e.g., L755507), Brefeldin A, a derivative thereof, an analog thereof, and a combination thereof.

In some embodiments, step (b) of the method further comprises administering to the subject a DNA replication enzyme inhibitor. Non-limiting examples of DNA replication enzyme inhibitors are described herein and include DNA ligase inhibitors (e.g., Scr7 or an analog thereof), DNA gyrase inhibitors, DNA helicase inhibitors, and combinations thereof.

In certain embodiments, administering a combination of the small molecule compound and the DNA replication enzyme inhibitor enhances the effect of the DNA nuclease to correct the mutation in the target gene compared to administering either the small molecule compound or the DNA replication enzyme inhibitor.

In some embodiments, step (a) of the method comprises administering to the subject via a delivery system selected from the group consisting of a nanoparticle, a liposome, a micelle, a virosome, a nucleic acid complex, and a combination thereof.

In some embodiments, step (b) of the method comprises administering to the subject via a delivery route selected from the group consisting of oral, intravenous, intraperitoneal, intramuscular, intradermal, subcutaneous, intra-arteriole, intraventricular, intracranial, intralesional, intrathecal, topical, transmucosal, intranasal, and a combination thereof.

In a fourth aspect, the present invention provides a system of identifying a small molecule compound to modulate genome editing of a target DNA in a cell, the system comprising:
(a) a first recombinant expression vector comprising a nucleotide sequence encoding a DNA nuclease or a variant thereof;
(b) a second recombinant expression vector comprising a nucleotide sequence encoding a DNA-targeting RNA operably linked to a promoter, wherein the nucleotide sequence comprises:
   (i) a first nucleotide sequence that is complementary to the target DNA; and
   (ii) a second nucleotide sequence that interacts with the DNA nuclease or the variant thereof; and
(c) a recombinant donor repair template comprising:
   (i) a reporter cassette comprising a nucleotide sequence encoding a reporter polypeptide; and
   (ii) two or more nucleotide sequences comprising two or more non-overlapping, homologous portions of the target DNA, wherein the nucleotide sequences are located at the 5′ and 3′ ends of the reporter cassette.

The system of identifying a small molecule compound to modulate genome editing of a target DNA in a cell can be used in ex vivo therapy. For example, the method to screen for a modulator of genome editing can be used to find a novel composition (e.g., small molecule) that can be used to enhance homologous recombination (e.g., in genomic engineering using a CRISPR/Cas system), which in turn can be used in ex vivo therapy (e.g., modifying cells with the novel composition found through the screening methods). For example, ex vivo therapy can comprise administering a composition (e.g., a cell) generated or modified outside of an organism to a subject (e.g., patient). In some embodiments, the composition (e.g., a cell) can be generated or modified by the method disclosed herein. In some embodiments, the composition (e.g., a cell) can be derived from the subject (e.g., patient) to be treated by the ex vivo therapy. In some embodiments, ex vivo therapy can include cell-based therapy, such as adoptive immunotherapy.

In some embodiments, the cell can comprise the first recombinant expression vector, the second recombinant expression vector, the recombinant donor repair template, or any combination thereof.

In some embodiments, the first recombinant expression vector comprises a DNA nuclease. The DNA nuclease can be selected from, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. For example, the DNA nuclease can be a Cas9 polypeptide, a variant thereof, or a fragment thereof. In some embodiments, the system also includes a cell. The cell can be a primary cell, including but not limited to, peripheral blood mononuclear cells (PBMC), peripheral blood lymphocytes (PBL), and other blood cell subsets. The cell can be an immune cell. The cell can be a T cell, a natural killer cell, a monocyte, a natural killer T cell, a monocyte-precursor cell, a hematopoietic stem cell or a non-pluripotent stem cell, a stem cell, or a progenitor cell. The cell can be a hematopoietic progenitor cell. The cell can be a human cell. The cell can be selected. The cell can be expanded ex vivo. The cell can be expanded in vivo. The cell can be CD45RO(−), CCR7(+), CD45RA(+), CD62L(+), CD27(+), CD28(+), or IL-7Rα(+). The cell can be autologous to a subject in need thereof. The cell can be non-autologous to a subject in need thereof. The cell can be a good manufacturing practices (GMP) compatible reagent. The cell can be a part of a combination therapy to treat cancer, infections, autoimmune disorders, or graft-versus-host disease (GVHD) in a subject in need thereof. In some embodiments, the system further comprises a library of small molecule compounds.

In some embodiments, the recombinant donor repair template is in a third recombinant expression vector. The recombinant donor repair template can comprise a reporter cassette comprising a nucleotide sequence encoding a reporter polypeptide and two or more nucleotide sequences comprising two or more non-overlapping, homologous portions of the target DNA, wherein the nucleotide sequences are located at the 5′ and 3′ ends of the reporter cassette. The nucleotide sequence encoding the reporter polypeptide can be operably linked to at least one nuclear localization signal. In other embodiments, the nucleotide sequence encoding the reporter polypeptide can be operably linked to a nucleotide sequence encoding a self-cleaving peptide. The self-cleaving peptide can be a viral 2A peptide, such as a E2A peptide, F2A peptide, P2A peptide, and T2A peptide. The reporter peptide of the recombinant donor repair template can be a detectable polypeptide, fluorescent polypeptide, or a selectable marker. For example, the reporter peptide of the recombinant donor repair template can be a superfolder GFP (sfGFP). The recombinant donor repair template can comprise two or more non-overlapping, homologous portions of the target DNA, wherein the nucleotide sequences are located at the 5′ and 3′ ends of the reporter cassette.

In some embodiments, the second recombinant expression vector of the system comprises at least two guide nucleic acids (e.g., DNA-targeting RNA), wherein each guide nucleic acid (e.g., DNA-targeting RNA) is directed to a different sequence of the target DNA. In some embodiments, the second recombinant expression vector of the system comprises a nucleotide sequence encoding a DNA-targeting RNA operably linked to a promoter, for example, inserted adjacent to or near a promoter. The promoter can be a ubiquitous, constitutive (unregulated promoter that allows for continual transcription of an associated gene), tissue-specific promoter or an inducible promoter. Expression of the nucleotide sequence encoding the guide nucleic acid (e.g., DNA targeting RNA) inserted adjacent to or near a promoter can be regulated. For example, the nucleotide sequence can be inserted near or next to a ubiquitous promoter. Some non-limiting examples of the ubiquitous promoter can be a CAGGS promoter, an hCMV promoter, a PGK promoter, an SV40 promoter, or a ROSA26 promoter. The promoter can also be endogenous or exogenous. For example, the nucleotide sequence encoding a DNA-targeting RNA can be inserted adjacent or near to an endogenous or exogenous ROSA26 promoter. Further, a tissue specific promoter or a cell-specific promoter can be used to control the location of expression. For example, the nucleotide sequence encoding a DNA-targeting RNA can be inserted adjacent or near to a tissue specific promoter. The tissue-specific promoter can be a FABP promoter, a Lck promoter, a CamKII promoter, a CD19 promoter, a Keratin promoter, an Albumin promoter, an aP2 promoter, an insulin promoter, an MCK promoter, an MyHC promoter, a WAP promoter, or a Col2A promoter. Inducible promoters can be used as well. These inducible promoters can be turned on and off when desired, by adding or removing an inducing agent. It is contemplated that an inducible promoter can be, but is not limited to, a Lac, tac, trc, trp, araBAD, phoA, recA, proU, cst-1, tetA, cadA, nar, PL, cspA, T7, VHB, Mx, and/or Trex.

In some embodiments, the nucleotide sequence comprises a first nucleotide sequence that is complementary to the target DNA and a second nucleotide sequence that interacts with the DNA nuclease or the variant thereof. The target DNA sequence can be complementary to a fragment (e.g. a guide sequence) of the guide nucleic acid (e.g., DNA targeting RNA) and can be immediately following by a protospacer adjacent motif (PAM) sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (e.g., be located 5′) a 5′NGG PAM, wherein N is any nucleotide, such that the guide sequence of the DNA-targeting RNA base pairs with the opposite strand to mediate cleavage at about 3 base pairs upstream of the PAM sequence. In some embodiments, the degree of complementarity between a guide sequence of the DNA-targeting RNA and its corresponding target DNA sequence, when optimally aligned using a suitable alignment algorithm, is about or more than about 50%, 55%, 60%, 65%, 70%, 75%, 80%, 85%, 90%, 91%, 92%, 93%, 94%, 95%, 96%, 97%, 98%, 99%, or more. The first nucleotide sequence that is complementary to the target DNA can comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end that can direct Cas9 to the target DNA site using RNA-DNA complementarity base pairing. In some embodiments, the first nucleotide sequence comprises, for instance, 20, 19, 18, 17, 16, 15, 14, 13, 12, 11, or 10 nucleic acids at the 5′ end that can direct Cas9 to the target DNA site using RNA-DNA complementarity base pairing. In other embodiments, the first nucleotide sequence comprises less than 20, e.g., 19, 18, 17, 16, 15, 14, 13, 12, 11, 10 or less, nucleic acids that are complementary to the target DNA site. In some instances, the first nucleotide sequence contains 1 to 10 nucleic acid mismatches in the complementarity region at the 5′ end of the targeting region. In other instances, the first nucleotide sequence contains no mismatches in the complementarity region at the last about 5 to about 12 nucleic acids at the 3′ end of the targeting region.

In some embodiments, the second nucleotide sequence that interacts with the DNA nuclease (e.g., Cas9) or the variant thereof can be a protein-binding sequence of the guide nucleic acid (e.g., DNA-targeting RNA). In some embodiments, the protein-binding sequence of the DNA-targeting RNA comprises two complementary stretches of nucleotides that hybridize to one another to form a double stranded RNA duplex (dsRNA duplex). The protein-binding sequence can be between about 30 nucleic acids to about 200 nucleic acids, e.g., about 40 nucleic acids to about 200 nucleic acids, about 50 nucleic acids to about 200 nucleic acids, about 60 nucleic acids to about 200 nucleic acids, about 70 nucleic acids to about 200 nucleic acids, about 80 nucleic acids to about 200 nucleic acids, about 90 nucleic acids to about 200 nucleic acids, about 100 nucleic acids to about 200 nucleic acids, about 110 nucleic acids to about 200 nucleic acids, about 120 nucleic acids to about 200 nucleic acids, about 130 nucleic acids to about 200 nucleic acids, about 140 nucleic acids to about 200 nucleic acids, about 150 nucleic acids to about 200 nucleic acids, about 160 nucleic acids to about 200 nucleic acids, about 170 nucleic acids to about 200 nucleic acids, about 180 nucleic acids to about 200 nucleic acids, or about 190 nucleic acids to about 200 nucleic acids. In certain aspects, the protein-binding sequence can be between about 30 nucleic acids to about 190 nucleic acids, e.g., about 30 nucleic acids to about 180 nucleic acids, about 30 nucleic acids to about 170 nucleic acids, about 30 nucleic acids to about 160 nucleic acids, about 30 nucleic acids to about 150 nucleic acids, about 30 nucleic acids to about 140 nucleic acids, about 30 nucleic acids to about 130 nucleic acids, about 30 nucleic acids to about 120 nucleic acids, about 30 nucleic acids to about 110 nucleic acids, about 30 nucleic acids to about 100 nucleic acids, about 30 nucleic acids to about 90 nucleic acids, about 30 nucleic acids to about 80 nucleic acids, about 30 nucleic acids to about 70 nucleic acids, about 30 nucleic acids to about 60 nucleic acids, about 30 nucleic acids to about 50 nucleic acids, or about 30 nucleic acids to about 40 nucleic acids.

In some embodiments, the first recombinant expression vector and the second recombinant expression vector are in a single expression vector.

In some embodiments, the system provided herein for modulating genome editing includes enhancing and/or decreasing (repressing) the efficiency of genome editing. In some instances, the genome editing is homology-directed repair (HDR) or nonhomologous end joining (NHEJ) of the target DNA. In certain embodiments, the small molecule compound enhances the efficiency of HDR, enhances the efficiency of NHEJ, decreases the efficiency of HDR, decreases the efficiency of NHEJ, or a combination thereof. In some instances, the small molecule compound enhances the efficiency of HDR of the target DNA and decreases the efficiency of NHEJ of the target DNA. In other instances, the small molecule compound enhances the efficiency of NHEJ of the target DNA and decreases the efficiency of HDR of the target DNA.

In a fifth aspect, the present invention provides a kit comprising the system described above and an instruction manual.

In a sixth aspect, the present invention provides a method for identifying a small molecule compound for modulating genome editing of a target DNA in a cell, the method comprising:
(a) introducing into a cell:
   (i) a first recombinant expression vector comprising a nucleotide sequence encoding a Cas9 polypeptide or a variant thereof,
   (ii) a second recombinant expression vector comprising a nucleotide sequence encoding a DNA-targeting RNA operably linked to a promoter, wherein the nucleotide sequence comprises a first nucleotide sequence that is complementary to a target DNA and a second nucleotide sequence that interacts with the Cas9 polypeptide or the variant thereof, and
   (iii) a recombinant donor repair template comprising a reporter cassette comprising a nucleotide sequence encoding a reporter polypeptide operably linked to a nucleotide sequence encoding a self-cleaving peptide, and two nucleotide sequences comprising two non-overlapping, homologous portions of the target DNA, wherein the nucleotide sequences are located at the 5′ and 3′ ends of the reporter cassette,
to generate a modified cell;
(b) contacting the modified cell with a small molecule compound;
(c) detecting the level of the reporter polypeptide in the modified cell; and
(d) determining that the small molecule compound modulates genome editing if the level of the reporter polypeptide is increased or decreased compared to its level prior to step (b).

In some embodiments, the recombinant donor repair template of the method is in a third recombinant expression vector. The nucleotide sequence encoding the reporter polypeptide can be operably linked to at least one nuclear localization signal. The self-cleaving peptide can be a viral 2A peptide, such as a E2A peptide, F2A peptide, P2A peptide, and T2A peptide. The reporter peptide of the recombinant donor repair template can be a fluorescent polypeptide.

In some embodiments, the second recombinant expression vector of the method comprises at least two DNA-targeting RNAs, wherein each DNA-targeting RNA is directed to a different sequence of the target DNA. The first recombinant expression vector and the second recombinant expression vector can be in a single expression vector.

In some embodiments, the method provided herein for modulating genome editing includes enhancing and/or decreasing (repressing) the efficiency of genome editing. In some instances, the genome editing comprises homology-directed repair (HDR) or nonhomologous end joining (NHEJ) of the target DNA. In certain embodiments, the small molecule compound enhances the efficiency of HDR, enhances the efficiency of NHEJ, decreases the efficiency of HDR, decreases the efficiency of NHEJ, or a combination thereof. In some instances, the small molecule compound enhances the efficiency of HDR of the target DNA and decreases the efficiency of NHEJ of the target DNA. In other instances, the small molecule compound enhances the efficiency of NHEJ of the target DNA and decreases the efficiency of HDR of the target DNA.

In some embodiments, the cell of the method is selected from the group consisting of a stem cell, human cell, mammalian cell, non-mammalian cell, vertebrate cell, invertebrate cell, plant cell, eukaryotic cell, bacterial cell, and archaeal cell.

A. Nucleases

The present invention includes using a DNA nuclease such as an engineered (e.g., programmable or targetable) DNA nuclease to induce genome editing of a target DNA sequence. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof.

In some embodiments, a nucleotide sequence encoding the DNA nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Depending on the target cell/expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

1. CRISPR/Cas System

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “single guide RNA” or “sgRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas nuclease has DNA cleavage activity. The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Casl, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. CRISPR-related endonucleases that are useful in the present invention are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenabacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of indel mutations at off-target DNA sites. Non-limiting examples of Cas9 nucleases or nickases are described in, for example, U.S. Pat. No. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983 or D986, as well as a mutation at H840 or N863. In some instances, the dCas9 enzyme contains a D10A or D10N mutation. Also, the dCas9 enzyme can include a H840A, H840Y, or H840N. In some embodiments, the dCas9 enzyme of the present invention comprises D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

For genome editing methods, the Cas nuclease can be a Cas9 fusion protein such as a polypeptide comprising the catalytic domain of the type IIS restriction enzyme, FokI, linked to dCas9. The FokI-dCas9 fusion protein (fCas9) can use two guide RNAs to bind to a single strand of target DNA to generate a double-strand break.

2. Zinc Finger Nucleases (ZFNs)

“Zinc finger nucleases” or “ZFNs” are a fusion between the cleavage domain of Fokl and a DNA recognition domain containing 3 or more zinc finger motifs. The heterodimerization at a particular position in the DNA of two individual ZFNs in precise orientation and spacing leads to a double-strand break in the DNA. In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that are useful in the present invention include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33.

3. TALENs

“TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of Fokl. Several mutations to FokI have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

4. Meganucleases

“Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some instances, the meganuclease is naturally-occurring (found in nature) or wild-type, and in other instances, the meganuclease is non-natural, artificial, engineered, synthetic, rationally designed, or man-made. In certain embodiments, the meganuclease of the present invention includes an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

B. Small Molecule Compounds

The present invention is based, in part, on the surprising discovery that small molecule compounds, such as a β adrenoceptor agonist (e.g., L755507) and Brefeldin A can improve knockin or HDR efficiency and/or inhibit knockout or NHEJ efficiency using nuclease-mediated genome editing methods such as the CRISPR/Cas system. Also, it was unexpectedly discovered that nucleoside analogs such as thymidine analogs (e.g., azidothymidine (AZT) and trifluridine (TFT)) can decrease knockin or HDR efficiency and/or increase knockout or NHEJ efficiency using nuclease-mediated genome editing methods such as the CRISPR/Cas system.

The term “β adrenoceptor agonist” or “β-adrenergic receptor agonist” refers to a compound, molecule, agent, or drug that can bind to a β1, β2 or β3 adrenoceptor and stimulate a response. Non-limiting examples of a β adrenoceptor agonist include L755507 (CAS 159182-43-1), abediterol, amibegron, arbutamine, arformoterol, arotinolol, bambuterol, befunolol, bitolterol, bromoacetylalprenololmenthane, broxaterol, buphenine, carbuterol, carmoterol, cimaterol, clenbuterol, denopamine, deterenol, dipivefrine, dobutamine, dopamine, dopexamine, ephedrine, epinephrine, etafedrine, etilefrine, ethylnorepinephrine, fenoterol, 2-fluoronorepinephrine, 5-fluoronorepinephrine, formoterol, hexoprenaline, higenamine, indacaterol, isoetarine, isoetherine, isoproterenol, isoprenaline, N-i sopropyloctopamine, isoxuprine, labetalol, levalbuterol, levonordefrin, levosalbutamol, mabuterol, metaproterenol, metaraminol, methoxyphenamine, methyldopa, norepinephrine, orciprenaline, olodaterol, oxyfedrine, phenylpropanolamine, pirbuterol, prenalterol, procaterol, pseudoephedrine, ractopamine, reproterol, rimiterol, ritodrine, salbutamol, salmeterol, sinterol, solabegron, terbulaline, tretoquinol, tulobuterol, vilanterol, xamoterol, zilpaterol, zinterol, LAS100977, PF-610355, L748337, BRL37344, a derivative thereof, an analog thereof, and a combination thereof.

Brefeldin A (BFA) is a macrocyclic lactone antibiotic synthesized from palmitate (C₁₆). Non-limiting examples of BFA analogs include BFA lactam, 6(R)-hydroxy-BFA, 7-dehydrobrefeldin A (7-oxo-BFA), and a combination thereof.

The term “nucleoside analog” refers to a compound, molecule, agent, or drug that is an analog of a pyrimidine (e.g., cytosine, uracil or thymine) or a purine (e.g., adenine or guanine). Non-limiting examples of a nucleoside analog include azidothymidine (AZT), trifluridine (trifluorothymidine or TFT), floxuridine (5-fluoro-2′-deoxyuridine (FdU)), idoxuridine, 5-fluorouracil, cytarabine (cytosine arabinoside), gemcitabine, didanosine (2′,3′-dideoxyinosine, ddI), zalcitabine (dideoxycytidine; 2′,3′-dideoxycytidine, ddC), stavudine (2′,3′-didehydro-2′,3′-dideoxythymidine, d4T), lamivudine (2′,3′-dideoxy-3′-thiacytidine, 3TC), abacavir, apricitabine, emtricitabine (FTC), entecavir, arabinosyl adenosine (Ara-A), fluorouracil arabinoside, mercaptopurine riboside, 5-aza-2′-deoxycytidine, arabinosyl 5-azacytosine, 6-azauridine, azaribine, 6-azacytidine, trifluoro-methyl-2′-deoxyuridine, thymidine, thioguanosine, 3-deazautidine, 2-chloro-2′-deoxyadenosine (2-CdA), 5-bromodeoxyuridine 5′-methylphosphonate, fludarabine (2-F-ara-AMP), 6-mercaptopurine, 6-thioguanine, 2-chlorodeoxyadenosine (CdA), 4′-thio-beta-D-arabinofuranosylcytosine, 8-amino-adenosine, acyclovir, adefovir dipivoxil, allopurinol, azacytidine, azathioprine, caffeine, capecitabine, cidofovir, cladribine, clofarabine, decitabine, didanosine, dyphylline, emtricitabine, entecavir, famcyclovir, flucytosine, fludarabine, floxuridine, gancyclovir, gemcitabine, lamivudine, mercaptopurine, nelarabine, penicyclovir, pentoxyfylline, pemetrexed, ribavirin, stavudine, telbivudine, tenofovir, theobromine, theophylline, thioguanine, trifluridine, valacyclovir, valgancyclovir, vidarabine, zalcitabine, zidovudine, pyrazolopyrimidine nucleoside, a salt thereof, a derivative thereof, and a combination thereof.

The small molecule described herein can be contacted with a cell undergoing nuclease-mediated genome editing such as CRISPR/Cas-based genome modification. The small molecule can be used at a concentration of about 0.01 μM to about 10 μM, e.g., about 0.01 μM to about 0.05 μM, about 0.01 μM to about 0.1 μM, about 0.01 μM to about 0.2 μM, about 0.01 μM to about 0.4 μM, about 0.01 μM to about 0.6 μM, about 0.01 μM to about 0.8 μM, about 0.01 μM to about 1 μM, about 0.01 μM to about 2 μM, about 0.01 μM to about 3 μM, about 0.01 μM to about 4 μM, about 0.01 μM to about 5 μM, about 0.01 μM to about 6 μM, about 0.01 μM to about 7 μM, about 0.01 μM to about 8 μM, about 0.01 μM to about 9 μM, about 0.1 μM to about 1 μM, about 0.1 μM to about 2 μM, about 0.1 μM to about 3 μM, about 0.1 μM to about 4 μM, about 0.1 μM to about 5 μM, about 0.1 μM to about 6 μM, about 0.1 μM to about 7 μM, about 0.1 μM to about 8 μM, about 0.1 μM to about 9 μM, about 0.1 μM to about 10 μM, about 0.5 μM to about 1 μM, about 0.5 μM to about 2 μM, about 0.5 μM to about 4 μM, about 0.5 μM to about 6 μM, about 0.5 μM to about 8 μM, about 0.5 μM to about 10 μM, about 1 μM to about 2 μM, about 1 μM to about 4 μM, about 1 μM to about 6 μM, about 1 μM to about 8 μM, about 1 μM to about 10 μM, about 2 μM to about 4 μM, about 2 μM to about 6 μM, about 2 μM to about 8 μM, about 2 μM to about 10 μM, about 4 μM to about 6 μM, about 4 μM to about 8 μM, about 4 μM to about 10 μM, about 6 μM to about 8 μM, about 6 μM to about 10 μM, or about 8 μM to about 10 μM. The small molecule can be used at a concentration of at least about 0.01 μM, e.g., at least about 0.02 μM, at least about 0.04 μM, at least about 0.06 μM, at least about 0.08 μM, at least about 0.1 μM, at least about 0.2 μM, at least about 0.4 μM, at least about 0.6 μM, at least about 0.8 μM, at least about 1 μM, at least about 2 μM, at least about 4 μM, at least about 6 μM, at least about 8 μM, or at least about 10 μM. The cells undergoing genome editing can be treated with the small molecule compound at about 0 to about 72 hours, e.g., about 0 to about 72 hours, about 0 to about 12 hours, about 0 to about 24 hours, about 0 to about 36 hours, about 0 to about 48 hours, about 0 to about 60 hours, about 12 to about 24 hours, about 12 to about 36 hours, about 12 to about 48 hours, about 12 to about 60 hours, about 12 to about 72 hours, about 24 to about 36 hours, about 24 to about 48 hours, about 24 to about 60 hours, about 24 to about 72 hours, about 36 to about 48 hours, about 36 to about 60 hours, about 36 to about 72 hours, about 48 to about 60 hours, about 48 to about 72 hours, or about 60 to about 72 hours, after the components of the nuclease-mediated genome editing method such as the CRISPR/Cas system are introduced into the cell. In some embodiments, the cell is contacted with the small molecule compound for about 1 to about 72 hours, e.g., for about 1 to about 12 hours, for about 1 to about 24 hours, for about 1 to about 36 hours, for about 1 to about 48 hours, for about 1 to about 60 hours, for about 1 to about 72 hours, for about 12 to about 24 hours, for about 12 to about 36 hours, for about 12 to about 48 hours, for about 12 to about 60 hours, for about 12 to about 72 hours, for about 24 to about 36 hours, for about 24 to about 48 hours, for about 24 to about 60 hours, for about 24 to about 72 hours, for about 36 to about 48 hours, for about 36 to about 72 hours, or for about 48 to about 72 hours.

In particular embodiments, the small molecule compounds of the present invention can be used to modulate genome editing using any CRISPR/Cas system including those that are commercially available from, e.g., Life Technologies, Sigma-Aldrich, Addgene, OriGene, Clontech, and those described in U.S. Pat. Nos. 8,697,359, 8,795,965, 8,865,406, 8,889,356, and 8,906,616, and U.S. Application Publication Nos. 2014/0068797, 2014/0342456, and 2014/0356959.

C. Donor Repair Template for HDR

Provided herein is a recombinant donor repair template comprising a reporter cassette that includes a nucleotide sequence encoding a reporter polypeptide (e.g., a detectable polypeptide, fluorescent polypeptide, or a selectable marker), and two homology arms that flank the reporter cassette and are homologous to portions of the target DNA (e.g., target gene or locus) at either side of a DNA nuclease (e.g., Cas9 nuclease) cleavage site. The reporter cassette can further comprise a sequence encoding a self-cleavage peptide, one or more nuclear localization signals, and/or a fluorescent polypeptide, e.g. superfolder GFP (sfGFP).

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

The donor repair template can be cloned into an expression vector. Conventional viral and non-viral based expression vectors known to those of ordinary skill in the art can be used.

In place of a recombinant donor repair template, a single-stranded oligodeoxynucleotide (ssODN) donor template can be used for homologous recombination-mediated repair. An ssODN is useful for introducing short modifications within a target DNA. For instance, ssODN are suited for precisely correcting genetic mutations such as SNPs. ssODNs can contain two flanking, homologous sequences on each side of the target site of Cas9 cleavage and can be oriented in the sense or antisense direction relative to the target DNA. Each flanking sequence can be at least about 10 base pairs (bp), e.g., at least about 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1 kb, 2 kb, 4 kb, or longer. In some embodiments, each homology arm is about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb. The ssODN can be at least about 25 nucleotides (nt) in length, e.g., at least about 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, 100 nt, 150 nt, 200 nt, 250 nt, 300 nt, or longer. In some embodiments, the ssODN is about 25 to about 50; about 50 to about 100; about 100 to about 150; about 150 to about 200; about 200 to about 250; about 250 to about 300; or about 25 nt to about 300 nt in length.

D. Target Cells

The present invention can be used to modulate genome editing of any target cell of interest. The target cell can be a cell from any organism, e.g., a bacterial cell, an archaeal cell, a cell of a single-cell eukaryotic organism, a plant cell (e.g., a rice cell, a wheat cell, a tomato cell, an Arabidopsis thaliana cell, a Zea mays cell and the like), an algal cell (e.g., Botryococcus braunii, Chlamydomonas reinhardtii, Nannochloropsis gaditana, Chlorella pyrenoidosa, Sargassum patens C. Agardh, and the like), a fungal cell (e.g., yeast cell, etc.), an animal cell, a cell from an invertebrate animal (e.g., fruit fly, cnidarian, echinoderm, nematode, etc.), a cell from a vertebrate animal (e.g., fish, amphibian, reptile, bird, mammal, etc.), a cell from a mammal, a cell from a human, a cell from a healthy human, a cell from a human patient, a cell from a cancer patient, etc. In some cases, the target cell treated by the method disclosed herein can be transplanted to a subject (e.g., patient). For instance, the target cell can be derived from the subject to be treated (e.g., patient).

Any type of cell may be of interest, such as a stem cell, e.g., embryonic stem cell, induced pluripotent stem cell, adult stem cell, e.g., mesenchymal stem cell, neural stem cell, hematopoietic stem cell, organ stem cell, a progenitor cell, a somatic cell, e.g., fibroblast, hepatocyte, heart cell, liver cell, pancreatic cell, muscle cell, skin cell, blood cell, neural cell, immune cell, and any other cell of the body, e.g., human body. The cells can be primary cells or primary cell cultures derived from a subject, e.g., an animal subject or a human subject, and allowed to grow in vitro for a limited number of passages. In some embodiments, the cells are disease cells or derived from a subject with a disease. For instance, the cells can be cancer or tumor cells. The cells can also be immoralized cells (e.g., cell lines), for instance, from a cancer cell line.

Primary cells can be harvested from a subject by any standard method. For instance, cells from tissues, such as skin, muscle, bone marrow, spleen, liver, kidney, pancreas, lung, intestine, stomach, etc., can be harvested by a tissue biopsy or a fine needle aspirate. Blood cells and/or immune cells can be isolated from whole blood, plasma or serum. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cells including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be skewed towards particular populations and phenotypes. For example, the T cells can be skewed to phenotypically comprise, CD45RO(−), CCR7(+), CD45RA(+), CD62L(+), CD27(+), CD28(+) and/or IL-7Ra(+). Suitable cells can be selected that comprise one of more markers selected from a list comprising: CD45RO(−), CCR7(+), CD45RA(+), CD62L(+), CD27(+), CD28(+) and/or IL-7Rα(+). Induced pluripotent stem cells can be generated from differentiated cells according to standard protocols described in, for example, U.S. Pat. Nos. 7,682,828, 8,058,065, 8,530,238, 8,871,504, 8,900,871 and 8,791,248, the disclosures are herein incorporated by reference in their entirety for all purposes.

In some embodiments, the target cell is in vitro. In other embodiments, the target cell is ex vivo. In yet other embodiments, the target cell is in vivo.

E. Introducing Components of Nuclease-Mediated Genome Editing into Cells

Methods for introducing polypeptides and nucleic acids into a target cell (host cell) are known in the art, and any known method can be used to introduce a nuclease or a nucleic acid (e.g., a nucleotide sequence encoding the nuclease, a DNA-targeting RNA (e.g., single guide RNA), a donor repair template for homology-directed repair (HDR), etc.) into a cell, e.g., a stem cell, a progenitor cell, or a differentiated cell. Non-limiting examples of suitable methods include electroporation, viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments, the components of nuclease-mediated genome editing can be introduced into a target cell using a delivery system. In certain instances, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct) and one or more nucleic acid components such as a DNA-targeting RNA and/or a donor repair template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

F. Methods for Assessing the Efficiency of Genome Editing

To functionally test the presence of the correct genomic editing modification, the target DNA can be analyzed by standard methods known to those in the art. For example, indel mutations can be identified by sequencing using the SURVEYOR® mutation detection kit (Integrated DNA Technologies, Coralville, IA) or the Guide-it™ Indel Identification Kit (Clontech, Mountain View, Calif.). Homology-directed repair (HDR) can be detected by PCR-based methods, and in combination with sequencing or RFLP analysis. Non-limiting examples of PCR-based kits include the Guide-it Mutation Detection Kit (Clontech) and the GeneArt® Genomic Cleavage Detection Kit (Life Technologies, Carlsbad, Calif.). Deep sequencing can also be used, particularly for a large number of samples or potential target/off-target sites.

In certain embodiments, the efficiency (e.g., specificity) of genome editing corresponds to the number or percentage of on-target genome cleavage events relative to the number or percentage of all genome cleavage events, including on-target and off-target events.

In some embodiments, the small molecule compounds described herein (alone or in combination with one or more DNA replication enzyme inhibitors) are capable of modulating (e.g., enhancing or inhibiting (repressing)) genome editing of a target DNA sequence. The genome editing can comprise homology-directed repair (HDR) (e.g., insertions, deletions, or point mutations) or nonhomologous end joining (NHEJ).

In certain embodiments, the nuclease-mediated genome editing efficiency of a target DNA sequence in a cell is enhanced by at least about 0.5-fold, 0.6-fold, 0.7-fold, 0.8-fold, 0.9-fold, 1-fold, 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, or greater in the presence of a small molecule compound described herein (alone or in combination with a DNA replication enzyme inhibitor) compared to the absence thereof (e.g., a control cell that has not been contacted with the small molecule compound). In some embodiments, the small molecule compounds described herein such as, e.g., β adrenoceptor agonists (e.g., L755507) and Brefeldin A, can enhance CRISPR-mediated HDR efficiency by at least about 3-fold for large fragment insertions and by at least about 9-fold for point mutations. In other embodiments, the small molecule compounds described herein such as, e.g., nucleoside analogs (e.g., azidothymidine (AZT)), can enhance CRISPR-mediated NHEJ efficiency by at least about 2-fold.

In certain other embodiments, the nuclease-mediated genome editing efficiency of a target DNA sequence in a cell is reduced by at least about 0.5-fold, 0.6-fold, 0.7-fold, 0.8-fold, 0.9-fold, 1-fold, 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, or greater in the presence of a small molecule compound described herein (alone or in combination with a DNA replication enzyme inhibitor) compared to the absence thereof (e.g., a control cell that has not been contacted with the small molecule compound). In some embodiments, the small molecule compounds described herein such as, e.g., nucleoside analogs (e.g., azidothymidine (AZT), trifluridine (TFT), etc.), can decrease CRISPR-mediated HDR efficiency by at least about 3-fold. In other embodiments, the small molecule compounds described herein such as, e.g., such as, e.g., β adrenoceptor agonists (e.g., L755507), can decrease CRISPR-mediated NHEJ efficiency by at least about 2-fold.

G. Applications of Small Molecule Compounds for Modulating Gene Editing

The small molecule compounds described herein and those identified using the system and method of the present invention can be used to modulate the efficiency of genome editing.

For example, the modulation can increase efficiency of genome editing. In some cases, the modulation can be a decrease in cellular toxicity. The compounds can be applied to targeted nuclease-based therapeutics of genetic diseases. Current approaches for precisely correcting genetic mutations in the genome of primary patient cells have been very inefficient (less than 1 percent of cells can be precisely edited). The small molecules provided herein can enhance the activity of gene editing and increase the efficacy of gene editing-based therapies. Since the small molecules function at physiological dosages and within a short time period, they may be used for in vivo gene editing of genes in subjects with a genetic disease. The small molecule compounds can be administered to a subject via any suitable route of administration and at doses or amounts sufficient to enhance the effect (e.g., improve the genome editing efficiency) of the nuclease-based therapy.

The diseases that may be treated by the method include, but are not limited to, sickle cell anemia, hemophilia, neoplasia, cancer, age-related macular degeneration, schizophrenia, trinucleotide repeat disorders, fragile X syndrome, prion-related disorders, amyotrophic lateral sclerosis, drug addition, autism, Alzheimer's disease, Parkinson's disease, cystic fibrosis, blood and coagulation disease or disorders, inflammation, immune-related diseases or disorders, metabolic diseases, liver diseases and disorders, kidney diseases and disorders, muscular/skeletal diseases and disorders (e.g., muscular dystrophy, Duchenne muscular dystrophy), neurological and neuronal diseases and disorders, cardiovascular diseases and disorders, pulmonary diseases and disorders, ocular diseases and disorders, and the like.

The small molecule compounds can be used to create transgenic organisms, such as transgenic animals, plants, and cells. Generation of transgenic organisms requires precise deletion, insertion, or mutation of the embryonic cells or zygotes. Due to the low efficiency, screening of embryos that contain the desired modifications has been very difficult, and is a highly inefficient and costly (both in time and money) process. By using compounds that enhance genome editing (e.g., even by two-fold), fewer embryos will need to be screened to identify those with the desired modification, thus reducing the cost of generating transgenic organisms. The small molecules can be used to decrease cellular toxicity.

H. Identifying Small Molecule Compounds that Modulate CRISPR/Cas9-Mediated Genome Editing

The CRISPR/Cas system of genome modification includes a Cas9 nuclease or a variant thereof, a DNA-targeting RNA (e.g., a single guide RNA or sgRNA) containing a guide sequence that targets Cas9 to the target genomic DNA and a scaffold sequence that interacts with Cas9 (e.g., tracrRNA), and optionally, a donor repair template. In some instances, a variant of Cas9 such as a Cas9 mutant containing one or more of the following mutations: D10A, H840A, D839A, and H863A, or a Cas9 nickase can be substituted for the Cas9 nuclease. The donor repair template can include a nucleotide sequence encoding a reporter polypeptide such as a fluorescent protein or an antibiotic resistance marker, and homology arms that are homologous to the target DNA and flank the site of gene modification. Alternatively, the donor repair template can be a ssODN.

1. Target DNA

In the CRISPR/Cas system, the target DNA sequence can be complementary to a fragment of the DNA-targeting RNA and can be immediately following by a protospacer adjacent motif (PAM) sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (e.g., be located 5′) a 5′NGG PAM, wherein N is any nucleotide, such that the guide sequence of the DNA-targeting RNA base pairs with the opposite strand to mediate cleavage at about 3 base pairs upstream of the PAM sequence.

In some embodiments, the degree of complementarity between a guide sequence of the

DNA-targeting RNA and its corresponding target DNA sequence, when optimally aligned using a suitable alignment algorithm, is about or more than about 50%, 55%, 60%, 65%, 70%, 75%, 80%, 85%, 90%, 91%, 92%, 93%, 94%, 95%, 96%, 97%, 98%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, Selangor, Malaysia), and ELAND (Illumina, San Diego, Calif.).

The target DNA site can be selected in a predefined genomic sequence (gene) using web-based software such as ZiFiT Targeter software (Sander et al., 2007, Nucleic Acids Res, 35:599-605; Sander et al., 2010, Nucleic Acids Res, 38:462-468), E-CRISP (Heigwer et al., 2014, Nat Methods, 11:122-123), RGEN Tools (Bae et al., 2014, Bioinformatics, 30(10):1473-1475), CasFinder (Aach et al., 2014, bioRxiv), DNA2.0 gNRA Design Tool (DNA2.0, Menlo Park, Calif.), and the CRISPR Design Tool (Broad Institute, Cambridge, Mass.). Such tools analyze a genomic sequence (e.g., gene or locus of interest) and identify suitable target site for gene editing. To assess off-target gene modifications for each DNA-targeting RNA, computationally predictions of off-target sites are made based on quantitative specificity analysis of base-pairing mismatch identity, position and distribution.

2. DNA-Targeting RNA

The guide nucleic acid provided herein can be a DNA-targeting RNA. The DNA-targeting RNA (e.g., single guide RNA or sgRNA) can comprise a nucleotide sequence that is complementary to a specific sequence within a target DNA (e.g., a guide sequence) and a protein-binding sequence that interacts with the Cas9 polypeptide or a variant thereof (e.g., a scaffold sequence or tracrRNA). The guide sequence of a DNA-targeting RNA can comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end that can direct Cas9 to the target DNA site using RNA-DNA complementarity base pairing. In some embodiments, the guide sequence of a DNA-targeting RNA comprises about 100 nucleic acids at the 5′ end that can direct Cas9 to the target DNA site using RNA-DNA complementarity base pairing. In some embodiments, the guide sequence comprises 20 nucleic acids at the 5′ end that can direct Cas9 to the target DNA site using RNA-DNA complementarity base pairing. In other embodiments, the guide sequence comprises less than 20, e.g., 19, 18, 17, 16, 15 or less, nucleic acids that are complementary to the target DNA site. The guide sequence can include 17 nucleic acids that can direct Cas9 to the target DNA site. In some instances, the guide sequence contains about 1 to about 10 nucleic acid mismatches in the complementarity region at the 5′ end of the targeting region. In other instances, the guide sequence contains no mismatches in the complementarity region at the last about 5 to about 12 nucleic acids at the 3′ end of the targeting region.

The protein-binding sequence of the DNA-targeting RNA can comprise two complementary stretches of nucleotides that hybridize to one another to form a double stranded RNA duplex (dsRNA duplex). The protein-binding sequence can be between about 30 nucleic acids to about 200 nucleic acids, e.g., about 40 nucleic acids to about 200 nucleic acids, about 50 nucleic acids to about 200 nucleic acids, about 60 nucleic acids to about 200 nucleic acids, about 70 nucleic acids to about 200 nucleic acids, about 80 nucleic acids to about 200 nucleic acids, about 90 nucleic acids to about 200 nucleic acids, about 100 nucleic acids to about 200 nucleic acids, about 110 nucleic acids to about 200 nucleic acids, about 120 nucleic acids to about 200 nucleic acids, about 130 nucleic acids to about 200 nucleic acids, about 140 nucleic acids to about 200 nucleic acids, about 150 nucleic acids to about 200 nucleic acids, about 160 nucleic acids to about 200 nucleic acids, about 170 nucleic acids to about 200 nucleic acids, about 180 nucleic acids to about 200 nucleic acids, or about 190 nucleic acids to about 200 nucleic acids. In certain aspects, the protein-binding sequence can be between about 30 nucleic acids to about 190 nucleic acids, e.g., about 30 nucleic acids to about 180 nucleic acids, about 30 nucleic acids to about 170 nucleic acids, about 30 nucleic acids to about 160 nucleic acids, about 30 nucleic acids to about 150 nucleic acids, about 30 nucleic acids to about 140 nucleic acids, about 30 nucleic acids to about 130 nucleic acids, about 30 nucleic acids to about 120 nucleic acids, about 30 nucleic acids to about 110 nucleic acids, about 30 nucleic acids to about 100 nucleic acids, about 30 nucleic acids to about 90 nucleic acids, about 30 nucleic acids to about 80 nucleic acids, about 30 nucleic acids to about 70 nucleic acids, about 30 nucleic acids to about 60 nucleic acids, about 30 nucleic acids to about 50 nucleic acids, or about 30 nucleic acids to about 40 nucleic acids.

An exemplary embodiment of a protein-binding sequence of the DNA-targeting RNA (e.g., tracrRNA) is 5′-GTT GGA ACC ATT CAA AAC AGC ATA GCA AGT TAA AAT AAG GCT AGT CCG TTA TCA ACT TGA AAA AGT GGC ACC GAG TCG GTG CTT TTT; SEQ ID NO: 33. Another exemplary embodiment of a tracrRNA is 5′-AAG AAA TTT AAA AAG GGA CTA AAA TAA AGA GTT TGC GGG ACT CTG CGG GGT TAC AAT CCC CTA AAA CCG CTT TT; SEQ ID NO: 34. Another exemplary embodiment of a tracrRNA is 5′-ATC TAA AAT TAT AAA TGT ACC AAA TAA TTA ATG CTC TGT AAT CAT TTA AAA GTA TTT TGA ACG GAC CTC TGT TTG ACA CGT CTG AAT AAC TAA AAA; SEQ ID NO: 35. Yet another exemplary embodiment of a tracrRNA is 5′-TGT AAG GGA CGC CTT ACA CAG TTA CTT AAA TCT TGC AGA AGC TAC AAA GAT AAG GCT TCA TGC CGA AAT CAA CAC CCT GTC ATT TTA TGG CAG GGT GTT TTC GTT ATT T; SEQ ID NO: 36. Yet another exemplary embodiment of a tracrRNA is 5′-TTG TGG TTT GAA ACC ATT CGA AAC AAC ACA GCG AGT TAA AAT AAG GCT TAG TCC GTA CTC AAC TTG AAA AGG TGG CAC CGA TTC GGT GTT TTT TTT; SEQ ID NO: 37.

The DNA-targeting RNA can be selected using any of the web-based software described above. Considerations for selecting a DNA-targeting RNA include the PAM sequence for the Cas9 polypeptide to be used, and strategies for minimizing off-target modifications. Tools, such as the CRISPR Design Tool, can provide sequences for preparing the DNA-targeting RNA, for assessing target modification efficiency, and/or assessing cleavage at off-target sites.

The nucleotide sequence encoding the DNA-targeting RNA can be cloned into an expression cassette or an expression vector. In some embodiments, the nucleotide sequence is produced by PCR and contained in an expression cassette. For instances, the nucleotide sequence encoding the DNA-targeting RNA can be PCR amplified and appended to a promoter sequence, e.g., a U6 RNA polymerase III promoter sequence. In other embodiments, the nucleotide sequence encoding the DNA-targeting RNA is cloned into an expression vector that contains a promoter, e.g., a U6 RNA polymerase III promoter, and a transcriptional control element, enhancer, U6 termination sequence, one or more nuclear localization signals, etc. In some embodiments, the expression vector is multicistronic or bicistronic and can also include a nucleotide sequence encoding a fluorescent protein, an epitope tag and/or an antibiotic resistance marker. In certain instances of the bicistronic expression vector, the first nucleotide sequence encoding, for example, a fluorescent protein, is linked to a second nucleotide sequence encoding, for example, an antibiotic resistance marker using the sequence encoding a self-cleaving peptide, such as a viral 2A peptide. 2A peptides including foot-and-mouth disease virus 2A (F2A); equine rhinitis A virus 2A (E2A); porcine teschovirus-1 2A (P2A) and Thoseaasigna virus 2A

(T2A) have high cleavage efficiency such that two proteins can be expressed simultaneously yet separately from the same RNA transcript.

Suitable expression vectors for expressing the DNA-targeting RNA are commercially available from Addgene, Sigma-Aldrich, and Life Technologies. The expression vector can be pLQ1651 (Addgene Catalog No. 51024) which includes the fluorescent protein mCherry. The expression vectors can also contain a sequence encoding Cas9 or a variant thereof. Non-limiting examples of such expression vectors include the pX330, pSpCas9, pSpCas9n, pSpCas9-2A-Puro, pSpCas9-2A-GFP, pSpCas9n-2A-Puro, GeneArt® CRISPR Nuclease OFP vector, the GeneArt® CRISPR Nuclease OFP vector, and the like.

3. Small Molecule Library

After the polynucleotides of the present invention have been introduced into the target cells, the resulting cells can be exposed to a library of small molecule compounds in order to identify an enhancer or repressor of genome editing. In some embodiments, small molecules can be screened to identify those that increase the efficiency of DSBs and/or HDR at a specific target locus in a particular cell type.

The cell can be subjected to the small molecules at any concentration that is not detrimental to the cell, e.g., does not induce cell death, necrosis, or apoptosis. The cells can be treated with about 0.01 μM to about 10 μM, e.g., about 0.01 μM to about 0.05 μM, about 0.01 μM to about 0.1 μM, about 0.01 μM to about 0.2 μM, about 0.01 μM to about 0.4 μM, about 0.01 μM to about 0.6 μM, about 0.01 μM to about 0.8 μM, about 0.01 μM to about 1 μM, about 0.01 μM to about 2 μM, about 0.01 μM to about 3 μM, about 0.01 μM to about 4 μM, about 0.01 μM to about 5 μM, about 0.01 μM to about 6 μM, about 0.01 μM to about 7 μM, about 0.01 μM to about 8 μM, about 0.01 μM to about 9 μM, about 0.1 μM to about 1 μM, about 0.1 μM to about 2 μM, about 0.1 μM to about 3 μM, about 0.1 μM to about 4 μM, about 0.1 μM to about 5 μM, about 0.1 μM to about 6 μM, about 0.1 μM to about 7 μM, about 0.1 μM to about 8 μM, about 0.1 μM to about 9 μM, about 0.1 μM to about 10 μM, about 0.5 μM to about 1 μM, about 0.5 μM to about 2 μM, about 0.5 μM to about 4 μM, about 0.5 μM to about 6 μM, about 0.5 μM to about 8 μM, about 0.5 μM to about 10 μM, about 1 μM to about 2 μM, about 1 μM to about 4 μM, about 1 μM to about 6 μM, about 1 μM to about 8 μM, about 1 μM to about 10 μM, about 2 μM to about 4 μM, about 2 μM to about 6 μM, about 2 μM to about 8 μM, about 2 μM to about 10 μM, about 4 μM to about 6 μM, about 4 μM to about 8 μM, about 4 μM to about 10 μM, about 6 μM to about 8 μM, about 6 μM to about 10 μM, or about 8 μM to about 10 μM. The small molecule can be used at a concentration of at least about 0.01 μM, e.g., at least about 0.02 μM, at least about 0.04 μM, at least about 0.06 μM, at least about 0.08 μM, at least about 0.1 μM, at least about 0.2 μM, at least about 0.4 μM, at least about 0.6 μM, at least about 0.8 μM, at least about 1 μM, at least about 2 μM, at least about 4 μM, at least about 6 μM, at least about 8 μM, or at least about 10 μM. of the small molecule. In some embodiments, the cell and test small molecule are admixed from about 0 to about 72 hours, e.g., about 0 to about 72 hours, about 0 to about 12 hours, about 0 to about 24 hours, about 0 to about 36 hours, about 0 to about 48 hours, about 0 to about 60 hours, about 12 to about 24 hours, about 12 to about 36 hours, about 12 to about 48 hours, about 12 to about 60 hours, about 12 to about 72 hours, about 24 to about 36 hours, about 24 to about 48 hours, about 24 to about 60 hours, about 24 to about 72 hours, about 36 to about 48 hours, about 36 to about 60 hours, about 36 to about 72 hours, about 48 to about 60 hours, about 48 to about 72 hours, or about 60 to about 72 hours, after the nucleic acids are introduced into the cell.

To identify small molecules that modulate genetic editing in pluripotent stem cells, an iPS cell or embryonic stem cell comprising the system described herein including a donor repair template comprising a GFP reporter cassette with a viral 2A sequence and a nuclear localization sequence can be treated on a small molecule library. If more cells treated with the test small molecule are GFP-positive than those untreated, the test small molecule may be an enhancer of HDR-mediated genome editing. If fewer cells treated with the test small molecule are GFP-positive than those untreated, the test small molecule may be a repressor of HDR-mediated genome editing.

The systems and methods provided herein can also be used to identify compounds that modulate gene editing in other cells types and target loci. If the knockin efficiency, i.e., HDR efficiency increases by about 0.5-fold, 0.6-fold, 0.7-fold, 0.8-fold, 0.9-fold, 1-fold, 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, or more after treatment with a test small molecule compound, it is determined that the small molecule compound can improve or enhance knockin efficiency. If the knockin efficiency decreases by about 0.5-fold, 0.6-fold, 0.7-fold, 0.8-fold, 0.9-fold, 1-fold, 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, or more after small molecule compound treatment, the small molecule compound may be a repressor of HDR-mediated repair.

I. Kits

In certain aspects, the present invention provides a kit comprising: (a) a DNA nuclease or a nucleotide sequence encoding the DNA nuclease as described herein; and (b) a small molecule compound as described herein that modulates genome editing of a target DNA in a cell. The kit may further comprise one of more of the following components as described herein: a DNA-targeting RNA (e.g., sgRNA) or a nucleotide sequence encoding the DNA-targeting RNA; a recombinant donor repair template; a DNA replication enzyme inhibitor; or a combination thereof. The nucleotide sequence encoding the DNA nuclease, the nucleotide sequence encoding the DNA-targeting RNA, and/or the recombinant donor repair template can be located in one or more expression vectors. The kit can further include a cell to be modified using the expression vectors described herein. In some embodiments, the expression vectors of the kit have been introduced into the cell. The kit can also include an instruction manual.

In particular embodiments, the kit of the present invention can include: (a) a DNA-targeting RNA (e.g., sgRNA) or a nucleotide sequence encoding the DNA-targeting RNA; (b) a Cas9 polypeptide or variant thereof or a nucleotide sequence encoding the Cas9 polypeptide or variant thereof; (c) a small molecule compound that modulates genome editing of a target DNA in a cell; and optionally (d) a recombinant donor repair template and/or (e) a DNA replication enzyme inhibitor. In some embodiments, the recombinant donor repair template includes two nucleotide sequences comprising two non-overlapping, homologous portions of the target DNA, wherein the nucleotide sequences are located at the 5′ and 3′ ends of a nucleotide sequence corresponding to the target DNA to undergo genome editing. In some embodiments, the small molecule compound comprises a β adrenoceptor agonist (e.g., L755507) or an analog thereof, Brefeldin A or an analog thereof, a nucleoside analog (e.g., azidothymidine (AZT), trifluridine (TFT), etc.), a derivative thereof, or a combination thereof. The kit can also include an instruction manual.

In certain other aspects, provided herein is a kit comprising a first recombinant expression vector that includes a polynucleotide sequence encoding a Cas9 polypeptide or a variant thereof, a second recombinant expression vector that includes a polynucleotide sequence encoding a single guide RNA that is operably linked to a promoter, and a recombinant donor repair template. The single guide RNA comprises a first polynucleotide sequence that is complementary to the preselected target DNA and a second polynucleotide sequence that interacts with the Cas9 polypeptide or variant thereof. The recombinant donor repair template includes a reporter cassette and two polynucleotide sequences comprising two non-overlapping homologous sequences of the target DNA from each side of the target insertion site. The reporter cassette may be flanked by the two polynucleotide sequences. The reporter cassette includes a polynucleotide sequence encoding a reporter polypeptide (e.g., a fluorescent protein, an enzyme or an antibiotic resistance marker) and a polynucleotide sequence encoding a self-cleaving peptide. In some embodiments, the sequence encoding a reporter polypeptide is operably linked to at least one, e.g., 1, 2, 3, 4, 5 or more, nuclear localization signals. The recombinant donor repair template can be located in an expression vector. The kit can further include a cell to be modified using the expression vectors described herein. In some embodiments, the expression vectors of the kit have been introduced into the cell. The kit can also include an instruction manual.

### V. EXAMPLES

The following examples are offered to illustrate, but not to limit, the claimed invention.

### Example 1

### Identification of Small Molecules Enhancing CRISPR-Mediated Genome Editing

This example describes a high-throughput chemical screening platform based on a recombinant CRISPR/Cas9 reporter system that can be used in a variety of target cells. This example also illustrates a method for identifying small molecules that can increase or decrease the efficiency of homology-directed repair mediated gene editing in the system. Finally, this example describes small molecules that can enhance gene knockout of non-homologous end joining upon Cas9 cleavage.

### Summary

The bacterial CRISPR/Cas9 system has emerged as an effective tool for the sequence-specific gene knockout through non-homologous end joining (NHEJ), but it remains inefficient to precisely edit the genome sequence. Here we develop a reporter-based screening approach for the high-throughput identification of chemical compounds that can modulate precise genome editing through homology-directed repair (HDR). Using our screening method, we have characterized small molecules that can enhance CRISPR-mediated HDR efficiency, 3-fold for large fragment insertions and 9-fold for point mutations. Interestingly, we have also observed that a small molecule that inhibits HDR can enhance indel mutations mediated by NHEJ. The identified small molecules function robustly in diverse cell types with minimal toxicity. The use of small molecules provides a simple and effective strategy that enhances precise genome engineering applications and facilitates the study of DNA repair mechanisms in mammalian cells.

### Introduction

The bacterial adaptive immune system CRISPR (clustered regularly interspaced palindromic repeats)-Cas (CRISPR associated protein) has been used for the sequence-specific editing of mammalian genomes (Barrangou et al., 2007, Science, 315, 1709-1712; Cong et al., 2013, Science, 339, 819-823; Mali et al., 2013, Science, 339, 823-826; Smith et al., 2014, Cell Stem Cell, 15, 12-13; Wang et al., 2013, Cell, 153, 910-918; Yang et al., 2013, Cell, 154, 1370-1379). The CRISPR system derived from Streptococcus pyogenes uses a Cas9 nuclease protein that complexes with a single guide RNA (sgRNA) containing a 20-nucleotide (nt) sequence for introducing site-specific double-strand breaks (Hsu et al., 2013, Nat. Biotech., 31, 827-832; Jinek et al., 2012, Science, 337, 816-821). Targeting of the Cas9-sgRNA complex to DNA is specified by base pairing between the sgRNA and DNA as well as the presence of an adjacent NGG PAM (protospacer adjacent motif) sequence (Marraffini and Sontheimer, 2010, Nature, 463, 568-571). The double-strand break occurs 3 bp upstream of the PAM site, which allows for targeted sequence modifications via alternative DNA repair pathways: either non-homologous end joining (NHEJ) that introduces frame shift insertion and deletion (indel) mutations that lead to loss-of-function alleles (Geurts et al., 2009, Science, 325, 433; Lieber and Wilson, 2010, Cell, 142, 496-496.e491; Sung et al., 2013, Nat. Biotech., 31, 23-24; Tesson et al., 2011, Nat. Biotech., 31, 23-24; Wang et al., 2014, Science, 343, 80-84), or homology-directed repair (HDR) that can be exploited to precisely insert a point mutation or a fragment of desired sequence at the targeted locus (Mazón et al., 2010, Cell, 142, 648.e641-648.e642; Wang et al., 2014, Science, 343, 80-84; Yin et al., 2014, Nat. Biotech., 32, 551-553).

To date, CRISPR-mediated gene knockout through NHEJ has worked efficiently. For example, the efficiency for knocking out a protein-coding gene has been reported to be 20% to 60% in mouse embryonic stem (ES) cells and zygotes (Wang et al., 2013, Cell, 153, 910-918; Yang et al., 2013, Cell, 154, 1370-1379). However, introduction of a point mutation or a sequence fragment directed by a homologous template has remained relatively inefficient (Mali et al., 2013, Science, 339, 823-826; Wang et al., 2013, Cell, 153, 910-918; Yang et al., 2013, Cell, 154, 1370-1379). A long and tedious screening process via cell sorting or selection, expansion and sequencing is often required to identify correctly edited cells. Improving CRISPR-mediated precise gene editing remains a major challenge.

It has been shown that small molecule compounds can modulate the DNA repair pathways (Hollick et al., 2003, Bioorg. Med. Chem. Lett., 13, 3083-3086; Rahman et al., 2013, Hum. Gene. Ther., 24, 67-77; Srivastava et al., 2012, Cell, 151, 1474-1487). However, it remains unclear whether small molecules could be used to enhance CRISPR-induced DNA repair via HDR. We thus sought to identify new small molecules that could enhance HDR to promote more efficient precise gene insertion or point mutation correction.

### Results

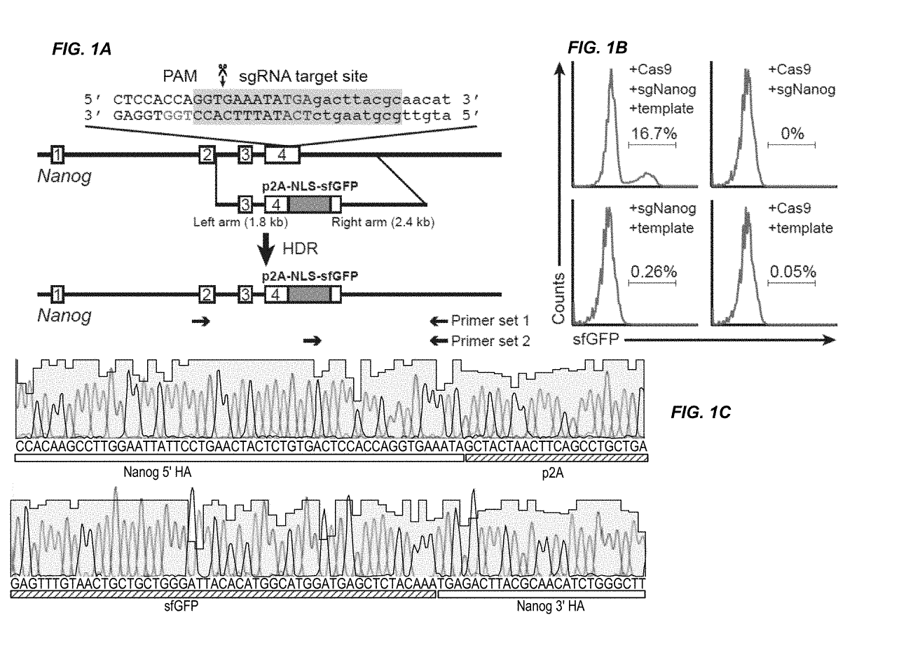

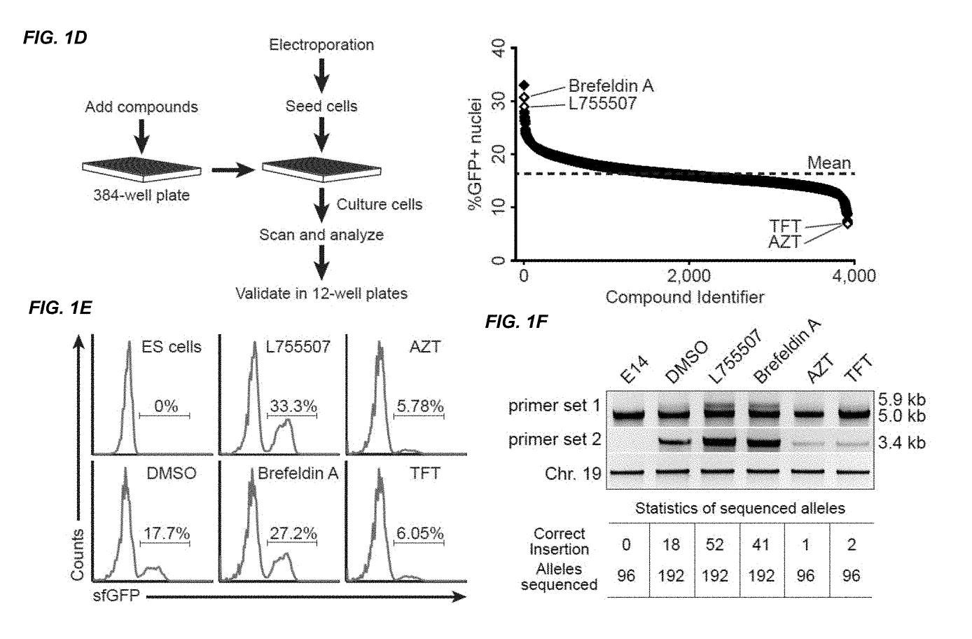

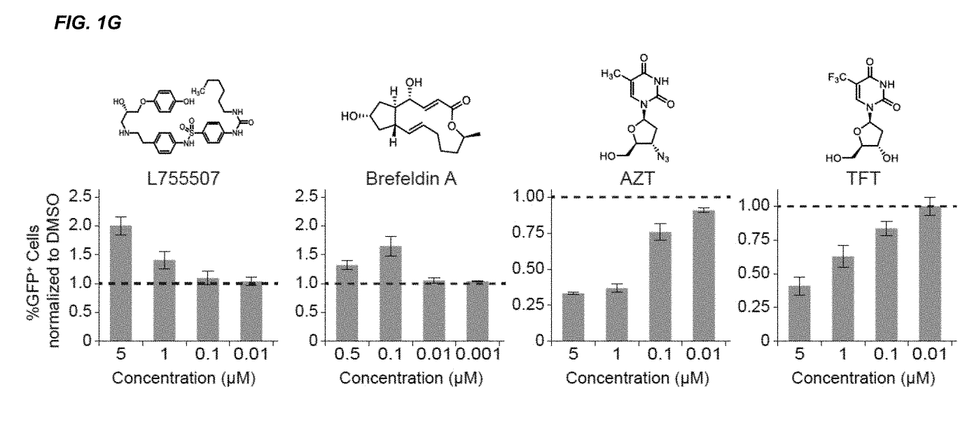

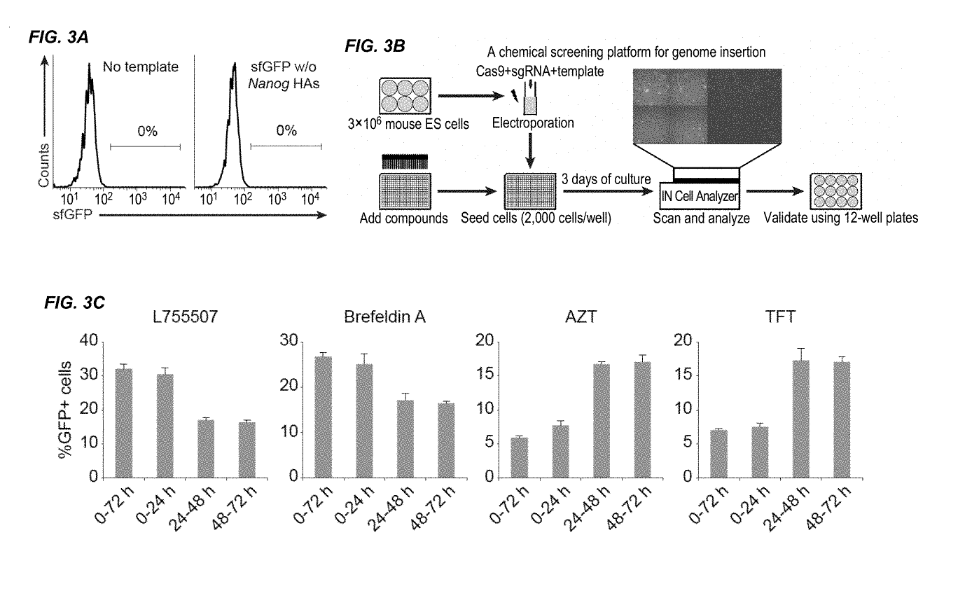

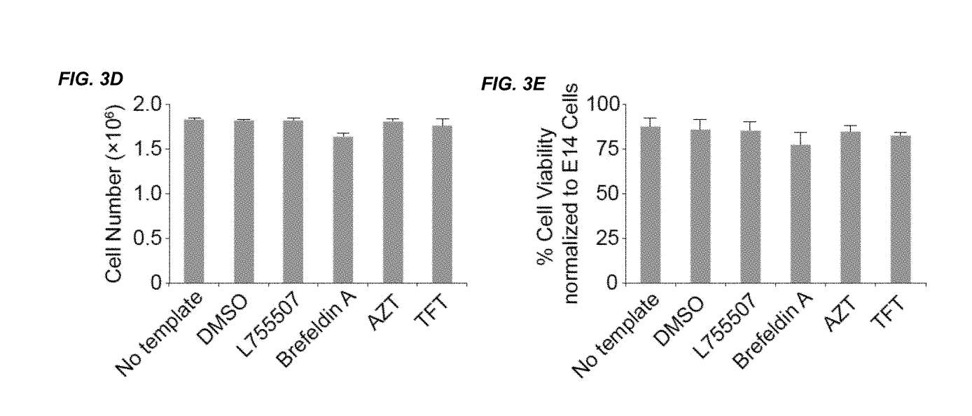

To characterize CRISPR-mediated HDR efficiency, we first established a fluorescence reporter system in E14 mouse ES cells. We used ES cells in the screening because compared to somatic cells, ES cells possess a decent HDR frequency, which provides a reasonable basal level of genome insertion (Kass et al., 2013, Proc Natl. Acad. Sci. USA, 110, 5564-5569). We co-transfected ES cells via electroporation with three plasmids: one expressing the nuclease Cas9, one expressing an sgRNA targeting the stop codon of the Nanog gene, and the third plasmid containing a promoterless superfolder GFP (sfGFP) with an in-frame N-terminal 2A peptide (p2A) and two nuclear localization sequences (NLSs) (FIG. 1A). The sfGFP cassette on the template is flanked by two homology arms to Nanog, a 1.8 kilo base (kb) left arm and a 2.4 kb right arm. CRISPR-induced in-frame insertion of the p2A-NLS-sfGFP sequence to the endogenous Nanog locus was detected by assessing green fluorescence using flow cytometry analysis 3 days post electroporation. Our results showed that only co-electroporation of all three plasmids generated GFP-positive ES cells (˜17% of cells showing strong fluorescence), while the controls lacking any of the three plasmids showed almost no GFP-positive cells (FIG. 1B). To confirm the correct insertion of template into the Nanog locus, we sorted GFP-positive cells, PCR amplified, and verified the target locus by sequencing. Our results showed correct HDR-mediated sfGFP integration in GFP-positive cells (FIG. 1C). Furthermore, we observed no fluorescence signal using a template without homology arms (FIG. 3A), suggesting a correlation between gain of fluorescence and HDR-mediated gene editing.

To investigate a broad range of small molecules that could act as enhancers or inhibitors of CRISPR-mediated HDR, we developed a high-throughput chemical screening assay based on the reporter system (FIGS. 1D and 3B). In this assay, mouse ES cells were co-transfected with Cas9, sgNanog, and the template, and seeded at 2,000 cells/well into Matrigel-coated 384-well plates containing the LIF-2i medium supplemented with individual compounds from our known drug collections. After 3 days of culture and chemical treatment, cells were fixed, stained with DAPI, and imaged by an automated high-content IN Cell imaging system to analyze the numbers of DAPI-positive and GFP/DAPI double-positive nuclei in each well.

From a collection of roughly 4,000 small molecules with known biological activity, we identified and subsequently confirmed using flow cytometry that two small molecules, L755507 and Brefeldin A, could improve the knockin efficiency (FIGS. 1D and 1E). L755507, a (33-adrenergic receptor agonist (Parmee et al., 1998, Bioorg. Med. Chem. Lett., 8, 1107-1112), increased the efficiency of GFP insertion by 3 fold compared to DMSO-treated control cells, which was further confirmed by PCR amplification and sequencing of the target locus and sequencing verification (FIGS. 1E and 1F). Brefeldin A, an inhibitor of intracellular protein transport from the endoplasmic reticulum to the Golgi apparatus (Ktistakis et al., 1992, Nature 356, 344-346), also improved insertion efficiency by 2-fold (FIGS. 1E and 1F).

Interestingly, we also identified two thymidine analogues, azidothymidine (AZT) and Trifluridine (TFT), that decreased the HDR efficiency (FIGS. 1D and 1E). AZT, previously used as an anti-HIV drug that inhibits the reverse transcriptase activity (Mitsuya et al., 1985, Proc. Natl. Acad. Sci. USA 82, 7096-7100), and TFT that was identified as an anti-herpesvirus drug by blocking viral DNA replication (Little et al., 1968, Proc. Soc. Exp. Biol. Med. 127, 1028-1032), showed decreased HDR efficiency by 3-fold assayed using flow cytometry (FIG. 1E), or by more than 10-fold assayed by sequencing (FIG. 1F).

We further examined the dosage effects, treatment duration, and cytotoxicity of identified small molecules. We found that HDR enhancers, L755507 and Brefeldin A, achieved their optimal enhancing effects at 5 μM and 0.1 μM, respectively (FIG. 1G). The HDR inhibitors, AZT and TFT, exhibited optimal inhibitory effects of knockin at 5 μM. In addition, we also examined compound treatment windows of 0-24 h, 24-48 h, 48-72 h, or 0-72 h post electroporation. All compounds showed optimal activity within the first 24 hours, suggesting that the genome knockin events occurred mostly during the first 24 hours in our system (FIG. 3C). Notably, at their optimized concentrations, the compounds exhibited no or very mild toxicity as assayed by both cell counts and MTS cell proliferation assay (FIGS. 3D and 3E).

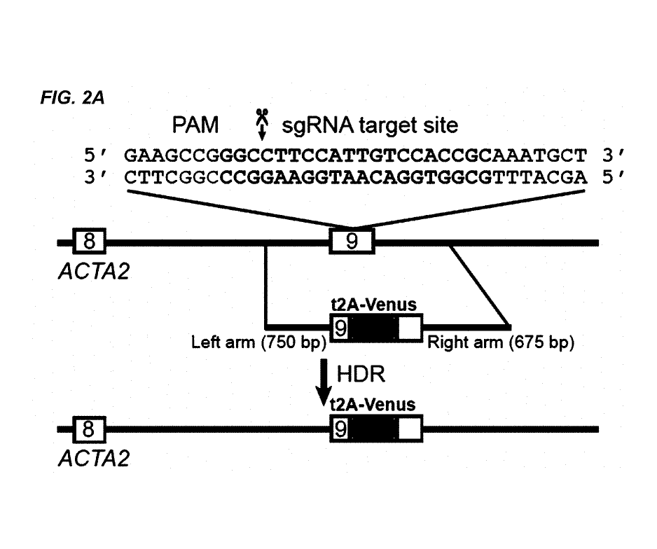

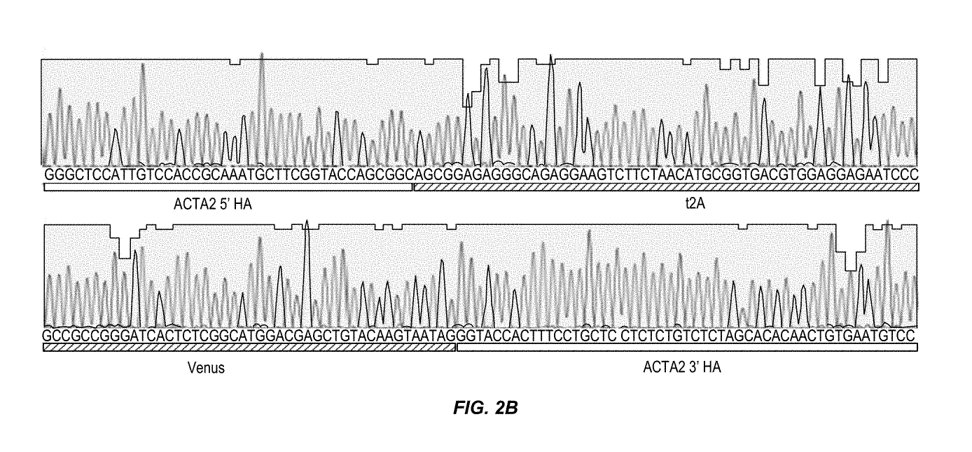

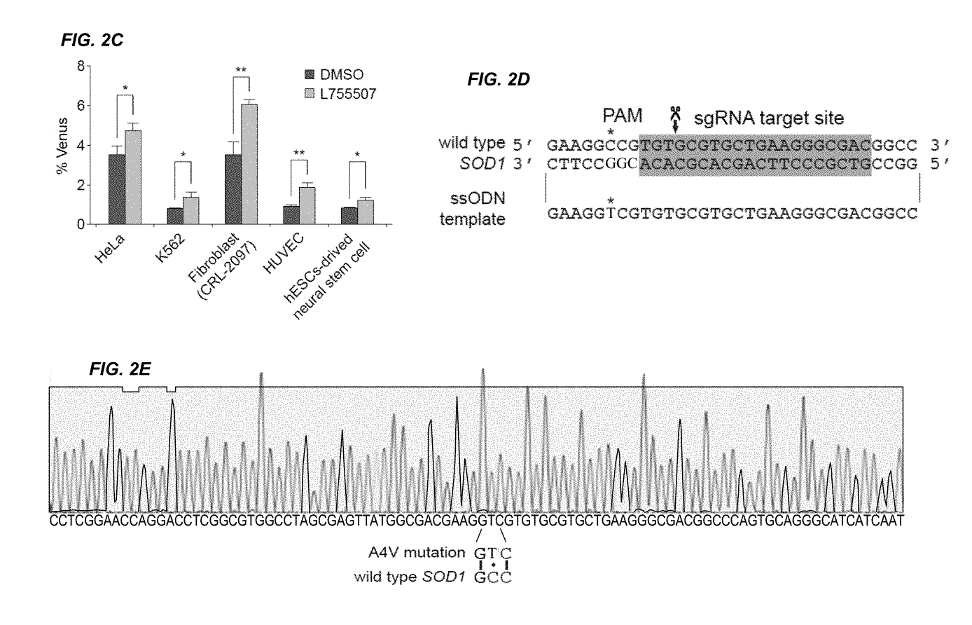

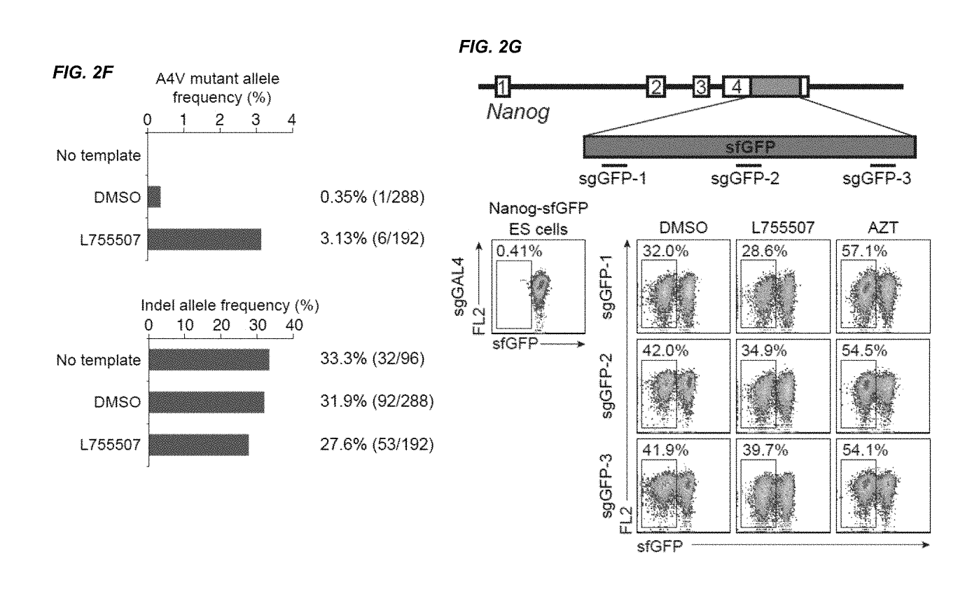

To test the generality of these compounds for modulating HDR at a different genomic locus, we used another template to insert a t2A-Venus cassette in frame into the Alpha Smooth Muscle Actin (ACTA2) locus (FIG. 2A), a gene expressed in a wide variety of cancer cell lines and normal cells (Ueyama et al., 1990, Jinrui idengaku zasshi, 35, 145-150). The template plasmid contains a left homology arm of 780 bp and a right homology arm of 695 bp that flank the t2A-Venus cassette. We first co-transfected the template plasmid with a single construct expressing both Cas9 and sgACTA2 into HeLa cells. Sequencing results of Venus-positive HeLa cells confirmed that Venus expression represented the correct insertion of Venus into the ACTA2 locus (FIG. 2B). We then tested several other types of human cells. Our flow cytometry results showed that the knockin efficiency was dependent on the cell type, ranging from 0.8% to 3.5%. Treating different types of cells with L755507 showed consistently improved HDR efficiency, with the largest increase of more than 2 fold in human umbilical vein endothelial cells (HUVEC). The fact that L755507 consistently increased the HDR efficiency in diverse cells including cancer cell lines (K562 and HeLa), suspension cells (K562), primary neonatal cells (HUVEC and fibroblast CRL-2097), and human ES cell-derived cells (neural stem cells) (Li et al., 2011, Proc Natl Acad Sci USA, 108, 8299-8304) suggested that the mechanism by which L755507 enhances CRISPR-mediated HDR is common in both transformed and primary cells

Precise editing of single-nucleotide polymorphisms (SNP) through single-stranded oligodeoxynucleotide (ssODN) templates is another important application of genome editing, with broad applications in disease modeling and gene therapy. We next sought to test whether the identified small molecule also enhanced SNP editing through HDR using a short ssODN. The method for introducing mutations into human pluripotent stem (iPS) cells using CRISPR-Cas9 and ssODN has been established (Ding et al., 2013, Cell Stem Cell, 12, 238-251; Yang et al., 2013, Nucleic Acids Res., 41, 9049-9061). Following a similar method, we synthesized a 200-nt ssODN template to introduce an A4V mutation into the human SOD1 locus (FIG. 2D), which is one of the common mutations that cause Amyotrophic Lateral Sclerosis (ALS) in the U.S. population (Rosen et al., 1994, Hum. Gene. Ther., 24, 67-77). We designed the sgRNA (sgSOD1) in a way that introduction of the A4V mutation also disrupted its PAM sequence, thus preventing further targeting by sgSOD1 of the A4V alleles. We co-transfected two vectors that encoded Cas9 and sgSOD1 with or without the ssODN template into human iPS cells (Ding et al., 2013, Cell Stem Cell, 12, 238-251; Ding et al., 2013, Cell Stem Cell, 12, 393-394; Zhu et al., 2010, Cell Stem Cell, 7, 651-655). The cells were then treated with DMSO or L755507 followed by genomic DNA extraction, PCR cloning and sequencing of randomly picked E. coli transformants. The sequencing results showed that compared to the DMSO control, L755507 enhanced the frequency of A4V allele mutant by almost 9-fold (FIGS. 2E and 2F). Our results also revealed reduced indel allele mutation frequency after the addition of L755507. These results demonstrate that our small molecules greatly enhanced SNP editing using a short ssODN template.

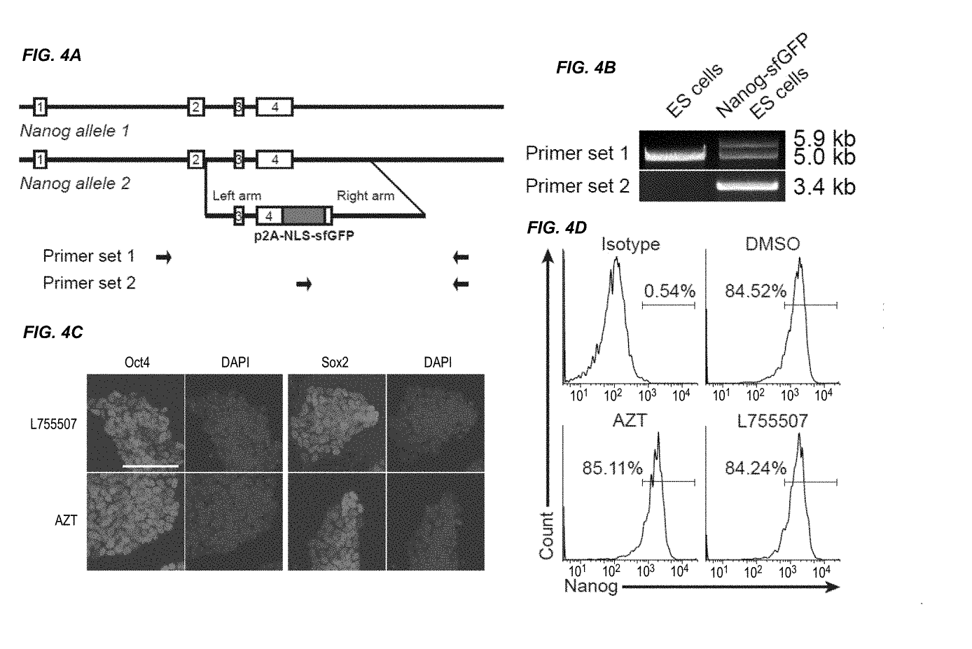

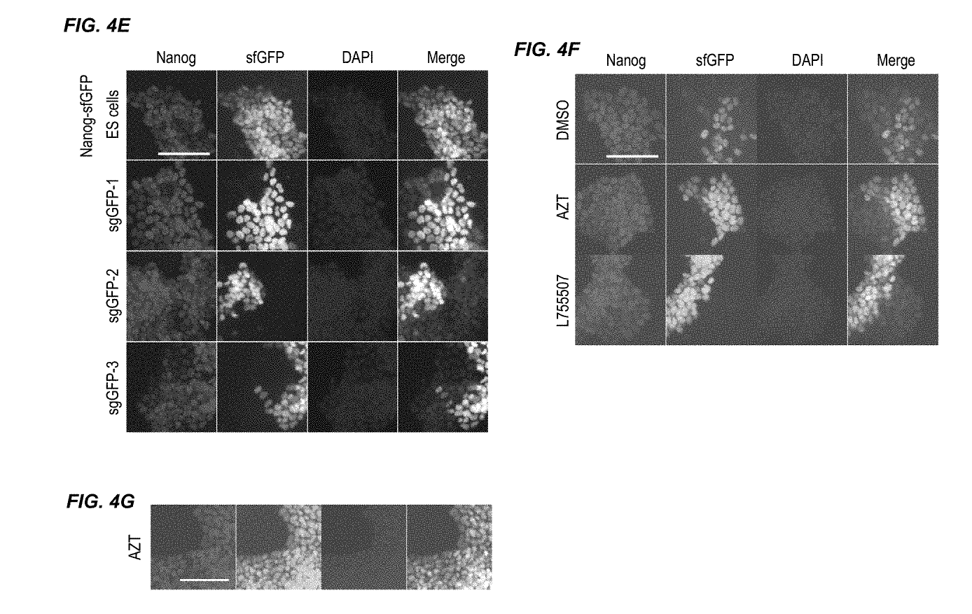

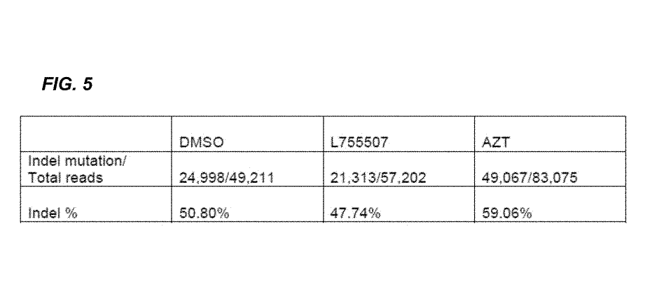

We then sought to test if the small molecules repressing HDR also affected NHEJ. We reasoned that if a small molecule directly inhibited the DNA cutting activity of Cas9, it should also inhibit CRISPR-mediated gene deletion without a template. To test this, we generated a clonal mouse ES cell line carrying a monoallelic sfGFP insertion at the Nanog locus (FIGS. 4A and 4B). We designed three sgRNAs (sgGFP-1, 2, 3) that targeted within the sfGFP coding sequence on the same plasmid that encoded Cas9 (FIG. 2G). Electroporation of any sgRNA resulted in a population of cells that showed complete loss of GFP expression after 3 days, while ES cells transfected with an sgRNA (sgGAL4) with no targetable sites showed no loss of the GFP signal (FIG. 2G). Adding L755507 to the cells immediately after electroporation showed inhibitory effects on GFP knockout. Unexpectedly, the knockin inhibitor, AZT, greatly increased GFP knockout efficiency for all three sgRNAs. For example, AZT increased the knockout efficiency by more than 1.8-fold in the case of sgGFP-1 (FIG. 2B). This was also consistent with the deep sequencing results for indel detection (FIG. 5). Together, these results suggest a possible trade-off between the NHEJ and HDR repair pathways.

Staining of three pluripotency markers Oct4, Sox2, and Nanog showed that the compounds did not affect cellular pluripotency (FIGS. 4C and 4D). Furthermore, neither electroporation (FIG. 4E) nor adding compounds (FIG. 4F) affected Nanog expression. The enhanced knockout efficiency suggests that AZT has acted on the NHEJ pathway instead of interacting with the Cas9-sgRNA complex. These results also showed that the compounds identified in the screening system could modulate CRISPR-mediated gene knockout. To rule out that the AZT does not cause more errors in replication that in turn lead to inactivation of EGFP, we passaged Nanog-sfGFP ES cells line for 10 passages under AZT treatment without the CRISPR system, and observed no loss of GFP signals (FIG. 4G).

In summary, we developed a high-throughput chemical screening platform for CRISPR genome editing and provided a proof-of-principle demonstration that small molecules could be used to modulate the efficiency of CRISPR-mediated precise gene editing. We report several small molecules that could enhance or repress HDR-mediated gene editing. The identified compounds might interact with factors that are involved in DNA repair pathways through NHEJ or HDR, thus providing a set of potentially useful tools for the mechanistic interrogation of these pathways. The identified chemicals also exhibit minimal toxicity and work in diverse cell types, and can be used to enhance both large template-mediated gene insertion and ssODN-mediated SNP editing. We also report small molecules that can enhance gene knockout without a template. The observation that reducing HDR could increase NHEJ might suggest a trade-off between the two DNA repair pathways after CRISPR DNA cutting. Identification of diverse classes of small molecules provides an approach that facilitates and accelerates CRISPR-mediated precise genome editing, which is useful for both biomedical research and clinical applications.

### Materials and Methods

Generation of sgRNA and DNA Template

To clone sgRNA mCherry vectors, the optimized sgRNA expression vector (pSLQ1651, Addgene Catalog No. 51024) was linearized via double digestion with BstXI and Xhol, and gel purified. New sgRNA sequences were PCR amplified from pSLQ1651 using different forward primers (see below) and a common reverse primer (sgRNA.R), digested with BstXI and XhoI, gel purified, and ligated to the linearized pSLQ1651 vector.

sgNanog.F (SEQ ID NO: 1):
GGAGA ACCAC CTTGT TGGCG TAAGT CTCAT
ATTTC ACCGT TTAAG AGCTA TGCTG GAAAC AGCA
sgSOD1.F (SEQ ID NO: 2):
GTATC CCTTG GAGAA CCACC TTGTT GGTCG
CCCTT CAGCA CGCAC AGTTT AAGAG CTATG CTGGA AACAG CA
sgRNA.R (SEQ ID NO: 3):
CTAGT ACTCG AGAAA AAAAG CACCG ACTCG
GTGCC AC

To clone a single Cas9-sgRNA expressing vector, the pX330 (Addgene catalog no. 42230) expression vector expressing Cas9 and sgRNA was linearized with Bbsl digestion, and gel purified. A pair of oligos for each targeting site were phosphorylated, annealed, and ligated to the linearized pX330.

sgsfGFP-1.F (SEQ ID NO: 4):
CACCG CATCA CCTTC ACCCT CTCCA
sgsfGFP-1.R (SEQ ID NO: 5):
AAACT GGAGA GGGTG AAGGT GATGC
sgsfGFP-2.F (SEQ ID NO: 6):
CACCG CGTGC TGAAG TCAAG TTTGA
sgsfGFP-2.R (SEQ ID NO: 7):
AAACT CAAAC TTGAC TTCAG CACGC
sgsfGFP-3.F (SEQ ID NO: 8):
CACCGTCGACAGGTAATGGTTGTC
sgsfGFP-3.R (SEQ ID NO: 9):
AAACG ACAAC CATTA CCTGT CGAC
sgACTA2.F (SEQ ID NO: 10):
CACCG CGGTG GACAA TGGAA GGCC
sgACTA2.R (SEQ ID NO: 11):
AAACG GCCTT CCATT GTCCA CCGC

The p2A-NLS-sfGFP template of Nanog was assembled from four DNA fragments, a 5′ homology arm, a p2A-NLS_{X2}-sfGFP cassette, a 3′ homology arm, and a modified pUC19 backbone vector, using Gibson Assembly Master Mix (New England Biolabs). Both 5′ and 3′ homology arms were PCR amplified from the genomic DNA extracted from mouse ES cells. The sequences of p2A and two copies of NLS were added to the upstream of sfGFP coding sequence by PCR amplification. The backbone vector was linearized by digestion with PmeI and ZraI. All DNA fragments were gel purified before the Gibson assembly reaction.

### Cell Culture, Electroporation, and Flow Cytometry Analysis

The E14 mouse ES cells were maintained in N2B27 medium (50% Neurobasal, 50% Dulbecco modified Eagle medium/Ham's nutrient mixture F12, 0.5% NEAA, 0.5% Sodium Pyruvate, 0.5% GlutaMax, 0.5% N2, 1% B27, 0.1mM β-mercaptoethanol and 0.05 g/L bovine albumin fraction V; all from Invitrogen) supplemented with LIF and 2i in gelatin-coated plates.

For electroporation, 3×10⁶ cells were electroporated using the Nucleofector Kit for Mouse Embryonic Stem Cells (Amaxa) with program A-030. For insertion experiments, 2.5 μg pX330 (Cas9), 2.5 μg sgNanog and 15 μg template (Nanog-p2A-NLS-sfGFP) were used. For sfGFP deletion experiments, 20 μg pX330 containing desired sgRNA was used. All plasmids were maxiprepped using the Endofree Maxiprep Kit (Qiagen). Cells post electroporation were counted with trypan blue, seeded to Matrigel-coated plates in LIF-containing ESGRO-2i medium (Millipore), and cultured for 3 days. At day 3, cells were analyzed using the BD FACSCalibur platform.

Human ES cell-derived neural stem cells were cultured in N2B27 medium supplemented with 3 μM of CHIR99021 and 1 μM of A-83-01. Human fibroblasts (CRL-2097) and HeLa cells were cultured in Dulbecco modified Eagle medium supplemented with 10% FBS (Gibco). K562 cells were cultured in RPMI medium supplemented with 10% FBS. HUVECs were culture using Endothelial Cell Growth Media Kit (Lonza). For insertion of Venus at the ACTA2 locus, 1×10⁷ cells were electroporated with 5 μg pX330-sgACTA2 and 15 μg template using the Neon Transfection System (Life Technologies). The programs used were: 1,300 V, 10 ms, and 3 pulses for human ES cell-derived neural stem cells; 1,500 V, 30 ms, and 1 pulse for fibroblasts; 1,005 V, 35 ms, and 2 pulses for HeLa; 1,450 V, 10 ms, and 3 pulses for K562; and 1,350 V, 30 ms, and 1 pulse for HUVEC. At day 3, cells were analyzed using the BD FACSCalibur platform.

### SOD1 SNP Editing in Human iPS Cells

The human induced pluripotent stem (iPS) cells (hiPSC-O#1, were cultured in mTeSR1 (STEMCELL Technologies) in Geltrex coated 6-well plates. Three hours prior electroporation, cells were moved to fresh mTeSR1 medium supplemented with 1 μM ROCK inhibitor (thiazovivin). Established method was used for the delivery of the Cas9 vector, sgSOD1 mCherry vector and the 200-nt ssODN template (SEQ ID NO: 12; 5′-GTGCT GGTTT GCGTC GTAGT CTCCT GCAGC GTCTG GGGTT TCCGT TGCAG TCCTC GGAAC CAGGA CCTCG GCGTG GCCTA GCGAG TTATG GCGAC GAAGG TCGTG TGCGT GCTGA AGGGC GACGG CCCAG TGCAG GGCAT CATCA ATTTC GAGCA GAAGG CAAGG GCTGG GACGG AGGCT TGTTT GCGAG GCCGC TCCCA-3′) (Ding et al., 2013, Cell Stem Cell 12, 238-251; Ding et al., 2013, Cell Stem Cell, 12, 393-394). Briefly, 1×10⁷ cells were electroporated with a mixture of 15 μg Cas9 vector, 15 μg sgSOD1 mCherry vector with or without (no template control) 30 μg ssODN template using the BioRad Gene Pulser. Cells were then recovered in mTeSR1 medium supplemented with 1 μM ROCK inhibitor with or without L755507 for 48 hours after electroporation. The mCherry positive cells were collected by Fluorescence Activated Cell Sorting (FACS) into 6-well plates and culture for 5 days before genome DNA preparation using PureLink Genomic DNA Mini Kit (Life Technologies). Genomic DNA was PCR amplified with Herculase II Fusion DNA polymerase (Agilent) using two primers flanking the homology arms (forward primer sequence: SEQ ID NO: 13; AAAGT GCCAC CTGAC AGGTC TGGCC TATAA AGTAG TCGCG; reverse primer sequence: SEQ ID NO: 14; AGCTG GAGAC CGTTT GACCC GCTCC TAGCA AAGGT). PCR products were purified using NucleoSpin Gel and PCR Cleanup Kit (Macherey-Nagel). The two primers contained extra 15-bp regions that allowed efficient subcloning onto a modified pUC19 vector using the In-Fusion HD Cloning Plus kit (Clontech). The cloning products were transformed into DH5αE. coli competent cells and grew on LB agar plates with Carbenicillin (Sigma). After overnight culture, we randomly picked 96, 288, and 192 colonies for no template, DMSO and L755507 samples, respectively. All E. coli colonies were minipreped and sequencing verified to detect the mutation sequences (QuintaraBio). The A4V allele mutant frequency is calculated as (# of A4V transformants)/(total # of bacterial transformants). The indel allele frequency is calculated as (# of indel transformants)/(total # of bacterial transformants). The allele that contained both A4V mutation and another indel was simply counted as an indel allele.

### Sequencing of Long Template Insertion of Nanog and ACTA2

For long template insertion at Nanog or ACTA2 loci, genomic DNA from 1×10⁶ cells were isolated and purified with PureLink Genomic DNA Mini Kit (Life Technologies). For sequencing, genomic DNA was PCR amplified with Herculase II Fusion DNA polymerase (Agilent) with a pair of primers outside homology arms. PCR products were purified and subcloned to a backbone vector (pUC19) using In-Fusion cloning for sequencing. The following PCR primers were used:

Nanog.F (SEQ ID NO: 15):
AAAGT GCCAC CTGAC ATTCT TCTAC CAGTC CCAAA CAAAA
GCTCTC
Nanog.R (SEQ ID NO: 16):
AGCTG GAGAC CGTTT AGCAA ATGTC AATCC CAAAG TTGGG
AG
ACTA2.F (SEQ ID NO: 17):
AAAGT GCCAC CTGAC CTGGT TAGCC AGTTT TCAC TGTTC
TCTGT
ACTA2.R (SEQ ID NO: 18):
AGCTG GAGAC CGTTT GCATT TTGGA AAGTC AAGAG GAGAG
AATTGC
For p2A-NLSx2-sfGFP insertion, a primer (SEQ ID
NO: 19; GCATG ACTTT TTCAA GAGTG CCA) that bound
within sfGFP was used to confirm correct insertion.

### Deep Sequencing of Nanog-sfGFP Knockout

For deep sequencing, the Nanog-sfGFP locus was PCR amplified and purified. Adapters and barcodes were added to amplicon by PCR. The DNA fragments were sequenced on a MiSeq (Illumina) with MiSeq Reagent Kit v3 (150 cycles) following the manufacturer's instructions.

Nanog-sfGFP-2.F (SEQ ID NO: 20):
ACACG TTCAG AGTTC TACAG TCCGA CGATC GACGG GACCT
ACAAG ACGCG
Nanog-sfGFP-2.R (SEQ ID NO: 21):
ACACG TTCAG AGTTC TACAG TCCGA CGATC GACGG GACCT
ACAAG ACGCG
5′ adapter primer (SEQ ID NO: 22):
AATGA TACGG CGACC ACCGA GATCT ACACG TTCAG AGTTC
TACAG TCCGA
3′ barcode primers:
(SEQ ID NO: 23)
CAAGC AGAAG ACGGC ATACG AGATA AACAG TGTGA
CTGGAGTTCC TTGGC ACCCG AGAAT TCCA;
(SEQ ID NO: 24)
CAAGC AGAAG ACGGC ATACG AGATA AACCC CGTGA
CTGGA GTTCC TTGGC ACCCG AGAAT TCCA;
(SEQ ID NO: 25)
CAAGC AGAAG ACGGC ATACG AGATA AACGG CGTGA
CTGGA GTTCC TTGGC ACCCG AGAAT TCCA.

### Small Molecule Compound Library and Screening

Sigma LOPAC library (1280 compounds), Tocriscreen library (1120 compounds), and part of Spectrum Collection library (1760 compounds) were screened. For screening, 50 nL/well of compound was added in Matrigel-coated 384-well plates containing 20 μL ESGRO-2i medium. After electroporation, 2,000 cells in 70 μL ESGRO-2i medium were seeded to the 384-well plates. After 3 days culture, cells were fixed, stained with DAPI, and imaged using IN Cell analyzer (GE). The numbers of DAPI-positive nuclei and DAPI/GFP double-positive nuclei were counted by IN cell analyzer. The ratio of double-positive nuclei and DAPI-positive nuclei was calculated and plotted from high to low as shown in FIG. 1D. Extreme outliers were individually examined and excluded if the results were due to severe cell death.

Generation of a Clonal Mouse ES Cell Line Carrying Monoallelic sfGFP Insertion at the Nanog Locus

The E14 mouse ES cells electroporated with a template plasmid (p2A-NLS-sfGFP) were cultured for 3 days and dissociated into single cells with Accutase (Life Technologies). Single GFP-positive cells were sorted and seeded to each wells of a Matrigel-coated 96-well plate with the FACS Aria II (BD). 7 days after sorting, clonal GFP-positive colonies were expanded as normal ES cells. A rabbit polyclonal antibody (abcam) was used for immunofluorescence staining of Nanog.

### Toxicity Assay

Cells were treated with small molecules at the first 24 hours post electroporation. Cell number was counted at day 3 post electroporation. Cell viability was measured by the MTS assay (Promega) following manufacturer's instructions.

### Example 2

### Enhancement of Genome Editing Using Combinations of Small Molecules

This example illustrates that the efficiency of precise genome editing observed with the small molecules identified in Example 1 can be further enhanced by using them in combination with a small molecule inhibitor of an enzyme involved in DNA replication such as a DNA ligase, DNA gyrase, or DNA helicase. For example, the DNA ligase inhibitor can be Scr7 (5,6-bis((E)-benzylideneamino)-2-thioxo-2,3-dihydropyrimidin-4(1H)-one) or an analog thereof.

### Results

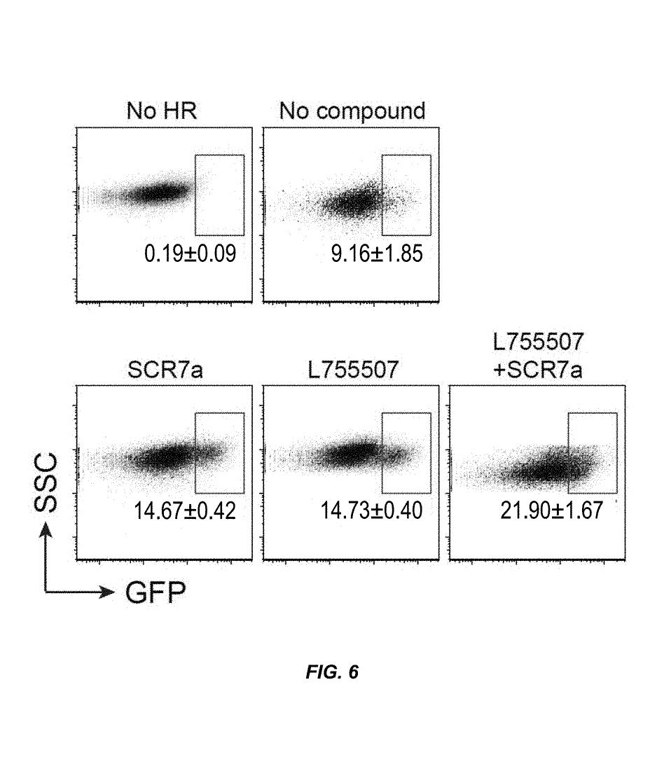

FIG. 6 shows the efficiency of GFP insertion using either a DNA ligase IV inhibitor such as an Scr7 analog (”SCR7a″) or a β3-adrenergic receptor agonist such as L755507, or a combination of both SCR7a and L755507. The combination of both SCR7a and L755507 enhanced the efficiency of homology-directed repair (HDR) as demonstrated by the increased percentage of GFP insertion over the use of either compound alone. The “No HR” control is ES cells only and the “No compound” control is DMSO only.

### Materials and Methods

### Cell Culture, Electroporation, and Flow Cytometry Analysis

The E14 mouse ES cells were maintained in N2B27 medium (50% Neurobasal, 50% Dulbecco modified Eagle medium/Ham's nutrient mixture F12, 0.5% NEAA, 0.5% Sodium Pyruvate, 0.5% GlutaMax, 0.5% N2, 1% B27, 0.1mM β-mercaptoethanol and 0.05 g/L bovine albumin fraction V; all from Invitrogen) supplemented with LIF and 2i in gelatin-coated plates.

For electroporation, 3×10⁶ cells were electroporated using the Nucleofector Kit for Mouse Embryonic Stem Cells (Amaxa) with program A-023. For insertion experiments, 2.5 μg pX330 (Cas9), 2.5 μg sgNanog and 15 μg template (Nanog-p2A-NLS-sfGFP) were used. For sfGFP deletion experiments, 20 μg pX330 containing desired sgRNA was used. All plasmids were maxiprepped using the Endofree Maxiprep Kit (Qiagen). Cells post electroporation were counted with trypan blue, seeded to Matrigel-coated plates in LIF-containing ESGRO-2i medium (Millipore), and cultured for 3 days. At day 3, cells were analyzed using the BD FACSCalibur platform.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

Informal Sequence Listing

SEQ ID NO: 1
sgNanog.F
GGAGA ACCAC CTTGT TGGCG TAAGT CTCAT ATTTC ACCGT
TTAAG AGCTA TGCTG GAAAC AGCA
SEQ ID NO: 2
sgSOD1.F
GTATC CCTTG GAGAA CCACC TTGTT GGTCG CCCTT CAGCA
CGCAC AGTTT AAGAG CTATG CTGGA AACAG CA
SEQ ID NO: 3
sgRNA.R
CTAGT ACTCG AGAAA AAAAG CACCG ACTCG GTGCC AC
SEQ ID NO: 4
sgsfGFP-1.F
CACCG CATCA CCTTC ACCCT CTCCA
SEQ ID NO: 5
sgsfGFP-1.R
AAACT GGAGA GGGTG AAGGT GATGC
SEQ ID NO: 6
sgsfGFP-2.F
CACCG CGTGC TGAAG TCAAG TTTGA
SEQ ID NO: 7
sgsfGFP-2.R
AAACT CAAAC TTGAC TTCAG CACGC
SEQ ID NO: 8
sgsfGFP-3.F
CACCGTCGACAGGTAATGGTTGTC
SEQ ID NO: 9
sgsfGFP-3.R
AAACG ACAAC CATTA CCTGT CGAC
SEQ ID NO: 10
sgACTA2.F
CACCG CGGTG GACAA TGGAA GGCC
SEQ ID NO: 11
sgACTA2.R
AAACG GCCTT CCATT GTCCA CCGC
SEQ ID NO: 12
ssODN template
5′-GTGCT GGTTT GCGTC GTAGT CTCCT GCAGC GTCTG GGGTT
TCCGT TGCAG TCCTC GGAAC CAGGA CCTCG GCGTG GCCTA
GCGAG TTATG GCGAC GAAGG TCGTG TGCGT GCTGA AGGGC
GACGG CCCAG TGCAG GGCAT CATCA ATTTC GAGCA GAAGG
CAAGG GCTGG GACGG AGGCT TGTTT GCGAG GCCGC
TCCCA-3′
SEQ ID NO: 13
forward primer for SOD1
AAAGT GCCAC CTGAC AGGTC TGGCC TATAA AGTAG TCGCG
SEQ ID NO: 14
reverse primer for SOD1
AGCTG GAGAC CGTTT GACCC GCTCC TAGCA AAGGT
SEQ ID NO: 15
Nanog.F
AAAGT GCCAC CTGAC ATTCT TCTAC CAGTC CCAAA CAAAA
GCTCTC
SEQ ID NO: 16
Nanog.R
AGCTG GAGAC CGTTT AGCAA ATGTC AATCC CAAAG TTGGG
AG
SEQ ID NO: 17
ACTA2.F
AAAGT GCCAC CTGAC CTGGT TAGCC AGTTT TCAC TGTTC
TCTGT
SEQ ID NO: 18
ACTA2.R
AGCTG GAGAC CGTTT GCATT TTGGA AAGTC AAGAG GAGAG
AATTGC
SEQ ID NO: 19
Primer for p2A-NLSx2-sfGFP insertion
GCATG ACTTT TTCAA GAGTG CCA
SEQ ID NO: 20
Nanog-sfGFP-2.F
ACACG TTCAG AGTTC TACAG TCCGA CGATC GACGG GACCT
ACAAG ACGCG
SEQ ID NO: 21
Nanog-sfGFP-2.R
ACACG TTCAG AGTTC TACAG TCCGA CGATC GACGG GACCT
ACAAG ACGCG
SEQ ID NO: 22
5′ adapter primer
AATGA TACGG CGACC ACCGA GATCT ACACG TTCAG AGTTC
TACAG TCCGA
SEQ ID NO: 23
3′ barcode primers
CAAGC AGAAG ACGGC ATACG AGATA AACAG TGTGA
CTGGAGTTCC TTGGC ACCCG AGAAT TCCA
SEQ ID NO: 24
3′ barcode primers
CAAGC AGAAG ACGGC ATACG AGATA AACCC CGTGA CTGGA
GTTCC TTGGC ACCCG AGAAT TCCA
SEQ ID NO: 25
3′ barcode primers
CAAGC AGAAG ACGGC ATACG AGATA AACGG CGTGA CTGGA
GTTCC TTGGC ACCCG AGAAT TCCA
SEQ ID NO: 26
5′-CTCCACCAGGTGAAATATGAGACTTACGCAACAT
SEQ ID NO: 27
5′-ATGTTGAGTAAGTCTCATATTTCACCTGGTGGAG
SEQ ID NO: 28
5′-GAAGCCGGGCCTTCCATTGTCCACCGCAAATGCT
SEQ ID NO: 29
5′-AGCATTTGCGGTGGACAATGGAAGGCCCGGCTTC
SEQ ID NO: 30
5′-GAAGGCCGTGGCGTGCTGCTGAAGGGCGACGGCC
SEQ IDNO: 31
5′-GGCCGTCGCCCTTCAGCACGCACACGGCCTTC
SEQ ID NO: 32
5′-GAAGGTCGTGTGTGCGTGCTGAAGGGCGACGGCC
SEQ ID NO: 33
tracrRNA
5′-GTT GGA ACC ATT CAA AAC AGC ATA GCA AGT TAA AAT
AAG GCT AGT CCG TTA TCA ACT TGA AAA AGT GGC ACC
GAG TCG GTG CTT TTT-3′
SEQ ID NO: 34
tracrRNA
5′-AAG AAA TTT AAA AAG GGA CTA AAA TAA AGA GTT TGC
GGG ACT CTG CGG GGT TAC AAT CCC CTA AAA CCG CTT
TT-3′
SEQ ID NO: 35
tracrRNA
5′-ATC TAA AAT TAT AAA TGT ACC AAA TAA TTA ATG CTC
TGT AAT CAT TTA AAA GTA TTT TGA ACG GAC CTC TGT
TTG ACA CGT CTG AAT AAC TAA AAA-3′
SEQ ID NO: 36
tracrRNA
5′-TGT AAG GGA CGC CTT ACA CAG TTA CTT AAA TCT TGC
AGA AGC TAC AAA GAT AAG GCT TCA TGC CGA AAT CAA
CAC CCT GTC ATT TTA TGG CAG GGT GTT TTC GTT ATT
T-3′
SEQ ID NO: 37
tracrRNA
5′-TTG TGG TTT GAA ACC ATT CGA AAC AAC ACA GCG AGT
TAA AAT AAG GCT TAG TCC GTA CTC AAC TTG AAA AGG
TGG CAC CGA TTC GGT GTT TTT TTT-3′

1. A method for modulating genome editing of a target DNA in a cell, the method comprising:
(a) introducing into the cell a DNA nuclease or a nucleotide sequence encoding the DNA nuclease, wherein the DNA nuclease is capable of creating a double-strand break in the target DNA to induce genome editing of the target DNA; and
(b) contacting the cell with a small molecule compound under conditions that modulate genome editing of the target DNA induced by the DNA nuclease.

2. The method of claim 1, wherein the modulating increases efficiency of genome editing.

3. The method of claim 1, wherein the modulating increases cell viability.

4. The method of claim 1, wherein the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof.

5. (canceled)

6. The method of claim 1, wherein step (a) further comprises introducing into the cell a DNA-targeting RNA or a nucleotide sequence encoding the DNA-targeting RNA.

7. (canceled)

8. The method of claim 1, wherein the small molecule compound that modulates genome editing is selected from the group consisting of a β adrenoceptor agonist or an analog thereof, Brefeldin A or an analog thereof, a nucleoside analog, a derivative thereof, and a combination thereof.

9. The method of claim 1, wherein the small molecule compound enhances or inhibits genome editing of the target DNA compared to a control cell that has not been contacted with the small molecule compound.

10. The method of claim 9, wherein the genome editing comprises homology-directed repair (HDR) of the target DNA.

11. The method of claim 10, wherein step (a) further comprises introducing into the cell a recombinant donor repair template.

12.-13. (canceled)

14. The method of claim 10, wherein the small molecule compound that enhances HDR is a β adrenoceptor agonist, Brefeldin A, a derivative thereof, an analog thereof, or a combination thereof.

15. The method of claim 14, wherein the β adrenoceptor agonist is L755507.

16. The method of claim claim 10, wherein the small molecule compound that inhibits HDR is a nucleoside analog, a derivative thereof, or a combination thereof.

17. The method of claim 16, wherein the nucleoside analog is azidothymidine (AZT), trifluridine (TFT), or a combination thereof

18. The method of claim 9, wherein the genome editing comprises nonhomologous end joining (NHEJ) of the target DNA.

19. The method of claim 18, wherein the small molecule compound that enhances NHEJ is a nucleoside analog or a derivative thereof

20. The method of claim 19, wherein the nucleoside analog is azidothymidine (AZT).

21. The method of claim 18, wherein the small molecule compound that inhibits NHEJ is a β adrenoceptor agonist or a derivative or analog thereof.

22. The method of claim 21, wherein the β adrenoceptor agonist is L755507.

23. The method of claim 1, wherein step (b) further comprises contacting the cell with a DNA replication enzyme inhibitor.

24. The method of claim 23, wherein the DNA replication enzyme inhibitor is selected from the group consisting of a DNA ligase inhibitor, a DNA gyrase inhibitor, a DNA helicase inhibitor, and a combination thereof.

25. The method of claim 23, wherein a combination of the small molecule compound and the DNA replication enzyme inhibitor enhances or inhibits genome editing of the target DNA compared to a control cell that has been contacted with either the small molecule compound or the DNA replication enzyme inhibitor.

26. The method of claim 25, wherein the genome editing comprises homology-directed repair (HDR) of the target DNA.

27. The method of claim 26, wherein the combination of the small molecule compound and the DNA replication enzyme inhibitor that enhances HDR is a combination of a β adrenoceptor agonist or a derivative or analog thereof and a DNA ligase inhibitor or a derivative or analog thereof.

28. The method of claim 27, wherein the β adrenoceptor agonist is L755507.

29. The method of claim 27, wherein the DNA ligase inhibitor is Scr7 (5,6-bis((E)-benzylideneamino)-2-thioxo-2,3-dihydropyrimidin-4(1H)-one) or an analog thereof.

30.-33. (canceled)

34. A kit comprising: (a) a DNA nuclease or a nucleotide sequence encoding the DNA nuclease; and (b) a small molecule compound that modulates genome editing of a target DNA in a cell.

35.-37. (canceled)

38. A method for preventing or treating a genetic disease in a subject, the method comprising:
(a) administering to the subject a DNA nuclease or a nucleotide sequence encoding the DNA nuclease in a sufficient amount to correct a mutation in a target gene associated with the genetic disease; and
(b) administering to the subject a small molecule compound in a sufficient amount to enhance the effect of the DNA nuclease.

39.-49. (canceled)
